SEQUENCE LISTING (7 sequences)

SEQ ID NO: 1 - RNA, 22 nt, Unknown
  Description of Unknown miR-146a oligonucleotide
ugagaacugaauuccauggguu

SEQ ID NO: 2 - RNA, 99 nt, Unknown
  Description of Unknown pre-miR-146a oligonucleotide
ccgauguguauccucagcuuugagaacugaauuccauggguugugucagugucagaccucugaaauucaguucuucagcugggauaucucugucaucgu

SEQ ID NO: 3 - DNA, 22 bp, Unknown
  Description of Unknown miR-146a coding oligonucleotide
tgagaactgaattccatgggtt

SEQ ID NO: 4 - DNA, 99 bp, Unknown
  Description of Unknown pre-miR-146a coding oligonucleotide
ccgatgtgtatcctcagctttgagaactgaattccatgggttgtgtcagtgtcagacctctgaaattcagttcttcagctgggatatctctgtcatcgt

SEQ ID NO: 5 - protein, 4 aa, Unknown
  Description of Unknown ′KDEL′ motif family peptide
KDEL

SEQ ID NO: 6 - RNA, 22 nt, Unknown
  Description of Unknown Notch-1 UTR oligonucleotide
caagaaaauaaacuaguucuca

SEQ ID NO: 7 - RNA, 21 nt, Unknown
  Description of Unknown ErbB4 UTR oligonucleotide
cccaugaaguacaauucucug

(19) United States
(12) Patent Application Publication
(10) Pub. No.: US 2016/0304959 A1
(43) Pub. Date: Oct. 20, 2016

(54) COMPOSITIONS AND METHODS FOR TREATING DIABETIC NEPHROPATHY

(71) Applicant: Rush University Medical Center, Chicago, IL (US)

(72) Inventors: Vineet GUPTA, Glenview, IL (US); Jochen REISER, Hinsdale, IL (US)

(21) Appl. No.: 15/104,870

(22) PCT Filed: Dec. 16, 2014
(86) PCT No.: PCT/US14/70479

§ 371 Date: Jun. 15, 2016

Related U.S. Application Data
(60) Provisional application No. 61/917,134, filed on Dec. 17, 2013.

(51) Int. Cl. C12Q 1/68

(52) U.S. Cl. CPC C12Q 1/6883; C12Q 2600/106; C12Q 2600/158; C12Q 2600/178

(57) ABSTRACT

This invention generally relates to methods and biomarkers for assessing a subject's susceptibility to developing diabetic nephropathy, or for assessing the progression of diabetic nephropathy in a subject. The invention also relates to methods of treating diabetic nephropathy, and methods for identifying a candidate agent for treating diabetic nephropathy.

### CROSS-REFERENCE TO RELATED APPLICATIONS

This application claims the benefit of U.S. Provisional Application No. 61/917,134, filed Dec. 17, 2013, the entire contents of which are incorporated herein by reference.

### REFERENCE TO SEQUENCE LISTING

This instant application contains a Sequence Listing which has been submitted in ASCII text file format via EFS-Web and is hereby incorporate by reference in its entirety. Said ASCII copy, created on Dec. 15, 2014, is named 006093-0458(PCT)_SL.txt and is 1,887 bytes in size.

### BACKGROUND OF THE INVENTION

Diabetic nephropathy (DN) is a major long-term complication of diabetes mellitus, and is the leading indication for dialysis and kidney transplantation in the United States (Marks and Raskin, 1998, Med Clin North Am, 82:877-907). The final stage of nephropathy is called end-stage renal disease, or ESRD. About 20-30% of patients with type 1 or type 2 diabetes develop evidence of nephropathy, but in type 2 diabetes, a considerably smaller fraction of progress to ESRD. However, because of the much greater prevalence of type 2 diabetes, such patients constitute over half of those diabetic patients currently starting on dialysis.

Diabetic nephropathy has no known cure. Treatment for diabetic nephropathy typically focuses on: slowing progression of the disease; relieving pain; and managing complications and restoring function. In many cases, diabetic nephropathy requires lifelong hemodialysis treatment. The need for regular hospital visits for time-consuming hemodialysis treatment interferes with patients' daily life. Therefore, it is beneficial to detect nephropathy early and delay its progression to ESRD.

Because not all diabetic patients develop diabetic nephropathy, there is a need to develop diagnostic methods and assays that can assess the likelihood of a diabetic patient to develop diabetic nephropathy, or methods and assays that can assess the progression of diabetic nephropathy. Further, therapeutic interventions and efforts may have the greatest impact if instituted very early in the course of the disease. Therefore, there is a need to identify diabetic patients who would benefit from aggressive treatment early on, if these patients have a strong likelihood of developing diabetic nephropathy, or a strong likelihood of rapidly progressing to ESRD.

### SUMMARY OF THE INVENTION

This invention generally relates to methods and biomarkers for assessing a subject's susceptibility to developing diabetic nephropathy, or for assessing the progression of diabetic nephropathy in a subject. The invention also relates to methods of treating diabetic nephropathy, and methods for identifying a candidate agent for treating diabetic nephropathy.

In one aspect, the invention provides a method of treating diabetic nephropathy, comprising: administering to a subject a therapeutically effective amount of a therapeutic agent selected from the group consisting of: an inhibitor of ErbB4, an inhibitor of Notch-1, miR-146a, a nucleic acid encoding miR-146a, a nucleic acid encoding pre-miR-146a, an miR-146a mimic, a pre-miR-146a mimic, an agent that can increase the level of miR-146a, and a combination thereof; wherein the subject has been diagnosed with diabetes or diabetes susceptibility, and wherein the subject has a low miR-146a level in the glomerular tissue and/or podocytes in comparison to a suitable control.

In certain embodiments, the subject is further characterized by (i) a high ErbB4 level in the glomerular tissue and/or podocytes, in comparison to a suitable control; and/or (ii) a high Notch-1 level in the glomerular tissue and/or podocytes in comparison to a suitable control.

In another aspect, the invention provides a method for treating diabetic nephropathy, comprising: administering to a subject who has diabetes or diabetes susceptibility, and who has a low miR-146a level in the glomerular tissue and/or podocytes in comparison to a suitable control, a therapeutically effective amount of a therapeutic agent selected from the group consisting of: an inhibitor of ErbB4, an inhibitor of Notch-1, miR-146a, a nucleic acid encoding miR-146a, a nucleic acid encoding pre-miR-146a, an miR-146a mimic, a pre-miR-146a mimic, an agent that can increase the level of miR-146a, and a combination thereof.

In certain embodiments, the subject is further characterized by (i) a high ErbB4 level in the glomerular tissue and/or podocytes, in comparison to a suitable control; and/or (ii) a high Notch-1 level in the glomerular tissue and/or podocytes in comparison to a suitable control.

In certain embodiments, the method comprises administering to the subject a therapeutically effective amount of an inhibitor of ErbB4. In certain embodiments, the method comprises administering to the subject a therapeutically effective amount of an inhibitor of Notch-1. In certain embodiments, the method comprises administering to said subject a therapeutically effective amount of miR-146a, a nucleic acid encoding miR-146a, or a nucleic acid encoding pre-miR-146a. In certain embodiments, the method comprises administering to said subject a therapeutically effective amount of an miR-146a mimic or a pre-miR-146a mimic. In certain embodiments, the method comprises administering to said subject a therapeutically effective amount of an agent that can increase the level of miR-146a.

In certain embodiments, the miR-146a level in the glomerular tissue of said subject is decreased about 50% or more, in comparison to a suitable control. In certain embodiments, the miR-146a level in podocytes of said subject is decreased about 50% or more, in comparison to a suitable control.

In certain embodiments, the subject is a human subject.

In certain embodiments, the miR-146a comprises SEQ ID NO: 1.

In another aspect, the invention provides a method for treating diabetic nephropathy comprising: (i) determining the level of miR-146a in a glomerular tissue or podocyte sample obtained from a subject having diabetes or diabetes susceptibility; and (ii) when the level of miR-146a in the tissue or podocyte sample of said subject is lower than the level in a suitable control, administering to said subject a therapeutically effective amount of a therapeutic agent selected from the group consisting of: an inhibitor of ErbB4, an inhibitor of Notch-1, miR-146a, a nucleic acid encoding miR-146a, a nucleic acid encoding pre-miR-146a, an miR-146a mimic, a pre-miR-146a mimic, an agent that can increase the level of miR-146a, and a combination thereof.

In certain embodiments, the method further comprises: determining the level of ErbB4 and/or Notch-1 in a glomerular tissue or podocyte sample obtained from said subject, wherein the subject is characterized by (i) a high ErbB4 level in the glomerular tissue or podocyte sample, in comparison to a suitable control; and/or (ii) a high Notch-1 level in the glomerular tissue or podocyte sample in comparison to a suitable control.

In another aspect, the invention provides a method for identifying a subject for treatment of diabetic nephropathy, comprising: determining the level of miR-146a in a glomerular tissue or podocyte sample obtained from a subject who has diabetes or diabetes susceptibility; wherein, when the level of miR-146a in the tissue or podocyte sample of said subject is lower than the level in the suitable control, the subject is a candidate for treatment of diabetic nephropathy using an agent selected from the group consisting of: an inhibitor of ErbB4, an inhibitor of Notch-1, miR-146a, a nucleic acid encoding miR-146a, a nucleic acid encoding pre-miR-146a, an miR-146a mimic, a pre-miR-146a mimic, an agent that can increase the level of miR-146a, and a combination thereof.

In certain embodiments, the method further comprises: determining the level of ErbB4 and/or Notch-1 in a glomerular tissue or podocyte sample obtained from said subject, wherein said subject is characterized by (i) a high ErbB4 level in the glomerular tissue or podocyte sample, in comparison to a suitable control; and/or (ii) a high Notch-1 level in the glomerular tissue or podocyte sample in comparison to a suitable control.

In certain embodiments, the method further comprises administering to said subject a therapeutically effective amount of an inhibitor of ErbB4. In certain embodiments, the method further comprises administering to said subject a therapeutically effective amount of an inhibitor of Notch-1. In certain embodiments, the method further comprises administering to said subject a therapeutically effective amount of miR-146a, a nucleic acid encoding miR-146a, or a nucleic acid encoding pre-miR-146a. In certain embodiments, the method comprises administering to said subject a therapeutically effective amount of an miR-146a mimic or a pre-miR-146a mimic. In certain embodiments, the method comprises administering to said subject a therapeutically effective amount of an agent that can increase the level of miR-146a.

In certain embodiments, the miR-146a level in a glomerular tissue sample from said subject is decreased about 50% or more, in comparison to a suitable control. In certain embodiments, the miR-146a level in a podocyte sample of said subject is decreased about 50% or more, in comparison to a suitable control.

In certain embodiments, the subject is a human subject.

In certain embodiments, the miR-146a comprises SEQ ID NO: 1.

The three biomarkers, miR-146a, ErbB4, Notch-1, described herein may be used singularly or in any combination to characterize subjects who would benefit from the treatment.

In another aspect, the invention provides a method for identifying a candidate agent for treating diabetic nephropathy, comprising: (i) providing a podocyte from a diabetic nephropathy subject, wherein the level of miR-146a in said podocyte is decreased, as compared to a suitable control; and (ii) contacting said podocyte with said candidate agent; wherein an increase in the level of miR-146a in the presence of said agent, as compared to the level of miR-146a in the absence of said agent, is indicative that said agent is useful for treating diabetic nephropathy. In certain embodiments, the miR-146a comprises SEQ ID NO: 1.

In another aspect, the invention provides a method for identifying a candidate agent for treating diabetic nephropathy, comprising: (i) providing a normal podocyte; (ii) contact said normal podocyte with a serum sample from a diabetic nephropathy subject; (iii) contacting said podocyte with said candidate agent; wherein an increase in the level of miR-146a in the presence of said agent, as compared to the level of miR-146a in the absence of said agent, is indicative that said agent is useful for treating diabetic nephropathy. In certain embodiments, the miR-146a comprises SEQ ID NO: 1.

In another aspect, the invention provides a kit for assessing a subject's susceptibility to developing diabetic nephropathy, or the progression of diabetic nephropathy in a subject, comprising: (i) a nucleic acid probe that hybridizes to miR-146a; and (ii) a detection agent for determining the level of miR-146a in one or more podocytes, or in a glomerular tissue sample. In certain embodiments, the miR-146a comprises SEQ ID NO: 1.

In certain embodiments, the detection agent comprises a fluorescent agent.

Also provided herein is the use of the therapeutic described herein for the treatment of diabetic nephropathy, and the use of the therapeutic described herein in the manufacture of a medicament for the treatment of diabetic nephropathy.

### BRIEF DESCRIPTION OF THE DRAWINGS

FIGS. 1A and 1B are schematic representations of proposed signal pathways of the biomarkers described herein. As shown in FIG. 1A, it is believed that diabetic mileu reduces miR-146a levels in podocytes, resulting in an up-regulation of Notch-1 and ErbB4 expression, and leading to podocyte injury and decline in renal function. Subjects with lower glomerular miR-146a expression also show faster regression in renal function. FIG. 1B shows exemplary treatment methods described herein. It is believed that miR-146a directly targets Notch-1 and ErbB4 in podocytes, which are up-regulated upon reduction in miR-146a levels, resulting in podocyte injury. Treatment with exogenous miR-146a or inhibitors of its downstream effectors, Notch-1 and ErbB4 pathways, can rescue the disease phenotype.

FIGS. 2A, 2B, and 2C show the primary sequence and secondary structure of mi-R146a. miRNA is transcribed in the nucleus as long primary miRNA (pri-miRNA), processed by endonuclease Drosha into precursor miRNA (pre-miRNA), and is exported into the cytoplasm. In the cytoplasm pre-miRNA is further cleaved by RNase Dicer to yield a mature 22-nt duplex miRNA. FIG. 2A shows the structure of pre-mir-146a (SEQ ID NO: 2), with 22-nt duplex highlighted. Typically, only one of the two strands of the 22-nt duplex act as a functional miRNA, which gets loaded into an Argonaut protein-based RNA-induced silencing complex (RISC) to suppress target gene expression. In miR-146a, this is typically the 5′-strand, as shown in FIG. 2B, also known as miR-146a-5p (SEQ ID NO: 1). FIG. 2C shows the sequence alignment of miR-146a target sites in Notch-1 (SEQ ID NOS 6 and 1, respectively, in order of appearance) and ErbB4 (SEQ ID NOS 7 and 1, respectively, in order of appearance) and ErbB4 mRNA 3′ UTRs (Bai et al, 2007; Halkein et al., 2013)).

FIGS. 3A, 3B, 3C, and 3D depict the expression of miR-146a in glomeruli and podocytes, and compare the kidneys of wild type (WT) animal subjects with miR-146a knock out (KO) kidneys. FIG. 3A provides representative images showing in situ hybridization with DIG-labeled anti-miR-146a LNA probes. The images show that miR-146a is expressed in glomeruli, in podocytes, in WT animals, and is absent in miR-146a KO kidneys. FIG. 3B provides representative images showing in situ hybridization with anti-miR-146a probes. The images show reduced miR-146a expression in kidney sections of DN animals (STZtreated DBA/2), as compared to the non-diabetic control (DBA/2). Labeling using scrambled control LNA probes confirms the selectivity of anti-miR-146a LNA probes towards their target. FIG. 3C is a histogram showing urinary albumin/creatinine ratio in B6 WT and miR-146a KO animals of various ages (shown in weeks), (n=1-4). FIG. 3D shows histochemical analyses using light microscopy and TEM examination of kidney sections of B6 WT and miR-146a KO animals of various ages (shown on the right, in months (m)). miR-146a KO animals show DN phenotype with increasing age. Representative PAS staining, TEM analyses, as well as staining with anti-WT-1 mAb, anti-Notch-1 mAb and anti-ErbB4 mAb are shown. The images show increased megangial sclerosis (PAS), podocyte foot process effacement and GBM-thickening (TEM), loss of WT-1 expression and podocyte numbers (WT-1), and increased expression of Notch-1 and ErbB4 in the glomeruli. Positive staining in podocytes is marked with an arrow (not all positive cells are labeled).

FIGS. 4A, 4B, 4C, and 4D depict urinary albumin levels in DN animals, and expression levels of mature miR-146a and Notch-1, Synpo, WT1, and Podocin mRNA. FIG. 4A is a bar graph showing urinary albumin/creatinine ratio in 12 wk old BTBR WT (control) and Ob−/Ob− (DN) animals (n=5-7). ***p<0.001. FIG. 4B shows the results of qRT-PCR based quantitation of expression levels of mature miR-146a miRNA, and Notch-1, Synpo, WT1, Podocin mRNA, as well as pre-mir-146a in kidney sections of 12 wk old BTBR WT (control) and BTBR Ob−/Ob− (DN) animals. FIG. 4C shows histochemical analyses of kidney sections from 12 week old BTBR WT and BTBR Ob−/Ob− animals. Representative H&E and PAS staining, as well as staining with anti-WT-1 mAb, anti-Notch-1 mAb and anti-ErbB4 mAb are shown. FIG. 4D shows immunohistochemical staining of kidney sections from DBA/2 (control) and STZ-treated DBA/2 animals 20 weeks post-STZ treatment. Representative images from staining with anti-Notch-1 mAb and anti-ErbB4 mAb are shown

FIGS. 5A, 5B, and 5C show that HG and TGFb reduce miR-146a levels while also increasing Notch-1 and ErbB4 in podocytes in vitro. FIG. 5A is a bar graph showing qRT-PCR based analysis of levels of various RNAs in podocytes cultured in normal glucose (5 mM) v/s treatment with HG (30 mM) for 2 h or 24 h (Notch-1). FIG. 5B is a bar graph showing qRT-PCR based analysis of levels of various RNAs in podocytes cultured in normal media or treated with TGFb in the absence or presence of ErbB4 inhibitor (JNJ) for 2 h. FIG. 5C provides representative immunofluorescence images showing podocytes cultured in normal media or treated with TGFb in the absence or presence of ErbB4 inhibitor (JNJ) for 2 h and stained for paxillin, synpo or ErbB4.

FIGS. 6A, 6B, 6C, and 6D demonstrate that down-regulation of miR-146a leads to increased albuminuria and faster progression to high albuminuria in DN subjects. FIG. 6A is a scatter plot showing correlation between relative expression of miR-146a v/s albuminuria in the isolated glomeruli of DN patient biopsies. Each dot represents an individual patient. Data are from two time-points; one at the time of biopsy (open circle, T1) and one at the end of observation period (5±1 yrs post-biopsy, closed circle T2). Relative miR146a level of 0.4 is shown to divide the plot into “Normal” v/s “Low” ranges of expression. FIG. 6B provides scatter plots showing correlation between NORMAL (>0.4, open circle) or LOW (<0.4, closed circle) levels of relative miR-146a expression v/s albuminuria from (A). Note that Y-axis scales are different in these two plots. The number of patients in each group and the student's t-test between the groups are also shown. FIG. 6C provides graphs showing disease progression in the two groups of patients in (A); (left) with relatively normal miR-146a expression levels (open circle) measured at the time of biopsy (T1) or at the end of observation period (T2), and (right) with reduced miR-146a expression levels (closed circle) measured at the same two time points. Significance of difference between the two sets was determined using student's t-test. FIG. 6D shows that miR-146a expression levels inversely correlate with levels of its direct target Notch-1. Scatter plot shows correlation between normal (>0.4, open circle) or reduced (<0.4, closed circle) levels of relative miR-146a expression v/s Notch-1 mRNA levels at the time of biopsy in the isolated glomeruli of DN patient biopsies. Each dot represents an individual patient. Significance of difference between the two sets was determined using student's t-test.

FIG. 7 show that Streptozotocin (STZ) treatment induced similar levels of hyperglycemia in both WT and miR-146a KO DN mice.

FIG. 8 demonstrates that miR-146a KO animals show increase in proteinuria starting at 6-8 wks when compared to the WT controls post STZ-treatment. The data shows that treating miR-146a KO+STZ mice with erlotinib significantly reduced proteinuria and protected the miR-146a KO DN animals from development of DN.

FIGS. 9A-9D are images of kidney sections analyzed using Transmission electron microscopy (TEM) to determine if erlotinib treatment protected podocytes from foot process effacement. FIGS. 9A and 9C show kidney sections for miR-146a KO DN mice or WT mice after STZ treatment. FIGS. 9B and 9D show kidney sections for miR-146a KO DN mice or WT mice after erlotinib treatment. The results show that erlotinib treatment significantly reduced podocyte foot process effacement in both groups of mice.

### DETAILED DESCRIPTION OF THE INVENTION

### 1. Overview

This invention generally relates to methods and biomarkers for assessing a subject's susceptibility to developing diabetic nephropathy, or for assessing the progression of diabetic nephropathy in a subject. The methods and biomarkers disclosed herein can be used to identify specific patient populations who are more likely to develop diabetic nephropathy (or have a fast regression of renal functions), and are more likely to benefit from early therapeutic interventions. The invention also relates to methods of treating diabetic nephropathy, and methods for identifying a candidate agent for treating diabetic nephropathy.

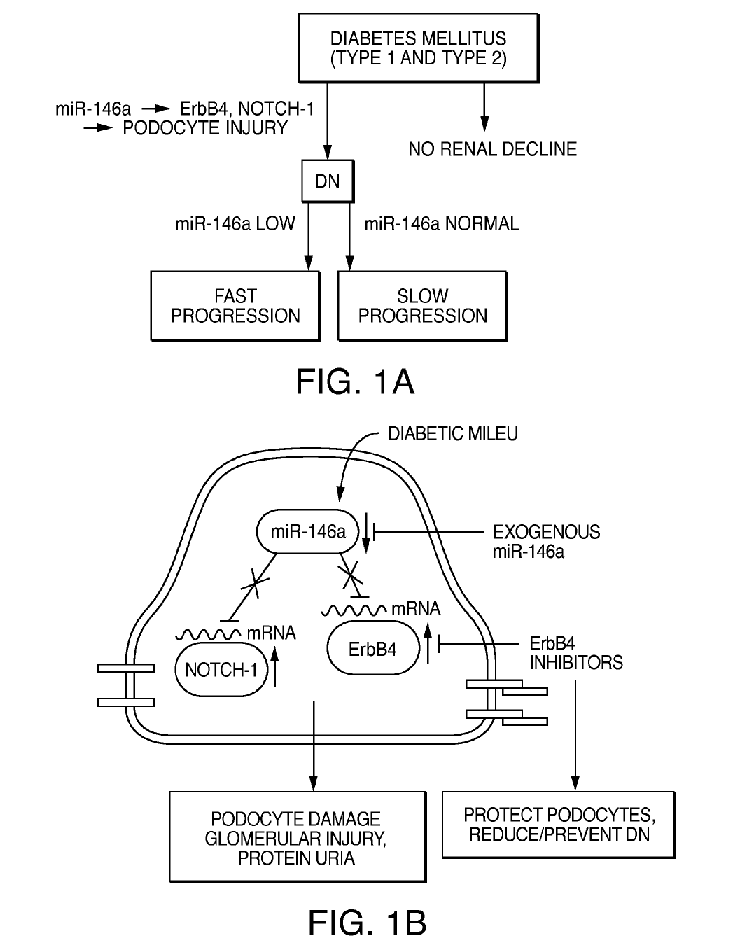

As described and exemplified herein, the inventors discovered that microRNA-146a (miR-146a) levels are significantly reduced in glomeruli of diabetic nephropathy (DN) subjects, as compared to diabetic subjects showing no signs of renal decline. Further, it was also found that reduced miR-146a levels correlate with a faster decline in renal functions in DN subjects, whereas in DN subjects that express normal level of miR-146a, a slow progression of renal decline was observed. Accordingly, miR-146a can be used as a biomarker for at least two purposes: (i) identifying diabetic subjects who are likely to develop renal diseases (e.g., diabetic nephropathy); and (ii) identifying diabetic subjects who have a strong likelihood of rapid regression of renal function (therefore, would likely benefit from aggressive treatment at an early stage of renal disease). See, FIGS. 1A and 1B.

The inventors also discovered that miR-146a directly inhibits the expression of two proteins, Notch-1 and ErbB4. Generally, the activities of Notch-1 and ErbB4 are not detected in normal, mature podocytes. In podocytes of DN subjects, however, the levels of Notch-1 and ErbB4 were increased. While not wishing to be bound by any particular theory, it is believed that increased expression of Notch-1 and ErbB4 is due to reduced miR-146a levels. It is believed that the increased expression and/or activities of Notch-1 and ErbB4 cause podocyte injury, and consequently, a decline in renal function. Therefore. Notch-1 and ErbB4 can also be used as a biomarker for identifying diabetic subjects who are likely to develop renal diseases (e.g., diabetic nephropathy); and identifying diabetic subjects who have a strong likelihood of rapid regression of renal function (therefore, would likely benefit from aggressive treatment at an early stage of renal disease).

Another aspect of the invention relates to the treatment of diabetic nephropathy. Therapeutic agents that can increase the level of miR-146a can be used to treat diabetic nephropathy. Such treatment can target specific diabetic subjects who have a reduced level of miR-146a. Additionally, therapeutic agents that can decrease the level (e.g., expression or activity level) of Notch-1 or ErbB4 can be also used to treat diabetic nephropathy. Such treatment can target specific diabetic subjects who have an increased level (e.g., expression or activity level) of Notch-1 or ErbB4.

Also provided herein are screening methods for identifying a candidate agent for treatment of diabetic nephropathy, based on the level (e.g., expression or activity level) of the biomarkers disclosed herein.

Also provided herein are kits for assessing a subject's susceptibility to developing diabetic nephropathy, or the progression of diabetic nephropathy in a subject, by determining the level of miR-146a.

### 2. Definitions

As used herein, the singular forms “a,” “an” and “the” include plural references unless the content clearly dictates otherwise.

The term “about”, as used here, refers to +/−10% of a value.

The terms “treat”, “treating”, and “treatment” refer to any manner in which one or more of the symptoms of a disease or disorder are beneficially altered, so as to prevent or delay the onset, prevent or delay the progression, or ameliorate the symptoms of a disease or disorder. The terms also include prophylactic treatment, such as decreasing the likelihood of developing diabetic nephropathy in a diabetic subject who has not been diagnosed with diabetic nephropathy or shows no symptoms of renal disease.

The term “normal podocyte” refers to a podocyte that does not exhibit a disease phenotype. The term “normal subject” as used herein refers to a subject who has not been diagnosed with a renal disease or diabetes.

### 3. Biomarkers for Diabetic Nephropathy

In one aspect, the invention provides biomarkers and methods for assessing a subject's susceptibility to developing diabetic nephropathy, or the progression of diabetic nephropathy in a subject. In particular, miR-146a, pre-miR146a, Notch-1, and ErbB-4 can be used as biomarkers for assessing the likelihood of developing diabetic nephropathy in a subject having diabetes or diabetes susceptibility, or the progression of renal decline in a subject diagnosed with diabetic nephropathy.

The biomarkers described herein can also be used to monitor the efficacy of a treatment, for example, by examining the biomarker levels periodically during the course of treatment.

In certain embodiments, the subject is a mammalian subject, preferable a human subject.

A. microRNA-146a

microRNA 146a (miR-146a) is a small non-coding RNA. microRNAs (miRNAs) are short (20-24 nt) non-coding RNAs that are involved in post-transcriptional regulation of gene expression in multicellular organisms by affecting both the stability and translation of mRNAs. In human, miR-146a is encoded by the MIR146A gene. miRNA genes are usually transcribed by RNA polymerase II (Pol II). The polymerase often binds to a promoter found near the DNA sequence encoding what will become the hairpin loop of the pre-miRNA. Animal miRNAs are initially transcribed as part of one arm of an about 80 nucleotide RNA stem-loop that in turn forms part of a several hundred nucleotides long miRNA precursor termed a primary miRNA (pre-miRNA). In the cytoplasm, the pre-miRNA hairpin is cleaved by the RNase III enzyme Dicer to produce mature miRNA.

Mature human miR-146a is 22 nucleotide long, as shown in SEQ ID NO: 1. Human pre-miR-146a is 99 nucleotide long, as shown in SEQ ID NO:2. DNA sequences encoding mature miR-146a and pre-miR-146a are provided in SEQ ID NOs: 3 and 4, respectively.

In one aspect, the invention provides a method for assessing a subject's susceptibility to developing diabetic nephropathy, or the progression of diabetic nephropathy in a subject, comprising determining the level of miR-146a in glomerulus tissue, and/or in one or more podocytes of said subject. A decreased level of miR-146a, as compared to a suitable control, indicates that the subject is susceptible to developing diabetic nephropathy, or is likely to have a fast progression of renal decline (e.g., fast progression to end-stage renal disease). In certain embodiments, the subject is diagnosed with diabetes.

A variety of controls may be used. For example, for comparison of miR-146a level in glomerulus tissue, a suitable control can be the level of miR-146a in a comparable glomerular tissue sample from a normal subject, or from the same subject before the subject is diagnosed with diabetes. For comparison of miR-146a in a podocyte, a suitable control can be miR-146a level in a normal podocyte (e.g., a podocyte from a standard cell line, or from a normal subject, or from the same subject before the subject is diagnosed with diabetes). Alternatively, a suitable control can be a pre-determined value, such as a known value from a database, or from prior investigations or reports, or population average.

In certain embodiments, a decrease of miR-146 level in podocytes or glomerular tissue of about 25% or more, relative to a suitable control, indicates that said subject is susceptible to developing diabetic nephropathy, or is likely to have a fast progression of renal decline. Preferably, the level of miR-146a is decreased by about 30% or more, about 35% or more, about 40% or more, about 45% or more, about 50% or more, about 55% or more, about 60% or more, about 65% or more, about 70% or more, about 75% or more, or about 80% or more, relative to a suitable control.

Fragments of mature miR-146a may also be used as biomarkers for DN. For example, “miRNA seed sequence” (or “seed region” or “seed portion”) typically refers to nucleotides 2-7 or 2-8 of the mature miRNA sequence. The miRNA seed sequence is typically located at the 5′ end of the miRNA. An exemplary miRNA-146a seed sequence is GAGAAC (nucleotides 2-7 of SEQ ID NO: 1).

In certain embodiments, miRNA molecules comprising SEQ ID NO. 1 or nucleotides 2-7 of SEQ ID NO: 1 are used as biomarkers for DN. Typically, an miRNA sequence comprises from about 6 to about 30 nucleotides, preferably about 22 nucleotides.

Variants of miR-146, such as microRNA molecules that are at least 85%, at least 90%, or at least 95% identical to SEQ ID NO: 1, may also be used as biomarkers for DN.

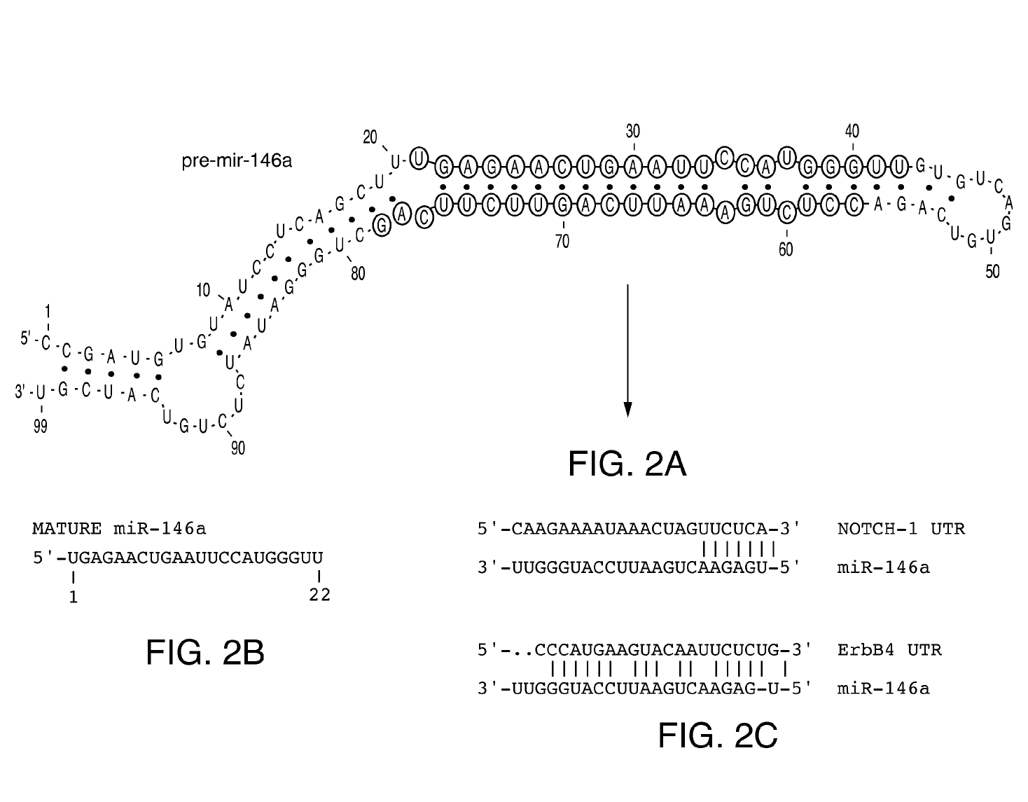

Useful fragments and variants include, e.g., a miRNA comprising the seeding region of miR-146a, or a miRNA comprising the sequence that binds to the 3′ UTR of Notch-1 or ErbB-4 (as shown in FIG. 2C).

pre-miR-146a may also be used as a biomarker. Interestingly, the inventors discovered that the level of pre-miR-146a is increased in podocytes or glomerular tissue samples of DN patients.

Accordingly, in another aspect, the invention provides a method for assessing a subject's susceptibility to developing diabetic nephropathy, or the progression of diabetic nephropathy in a subject, comprising determining the level of pre-miR-146a in glomerulus tissue, and/or in one or more podocytes of said subject. An increased level of pre-miR-146a, as compared to a suitable control, indicates that the subject is susceptible to developing diabetic nephropathy, or is likely to have a fast progression of renal decline (e.g., fast progression to end-stage renal disease). In certain embodiments, the subject is diagnosed with diabetes.

A suitable control for comparison of pre-miR-146a level in glomerular tissue can be the level of pre-miR-146a in a comparable glomerular tissue sample from a normal subject, or from the same subject before the subject is diagnosed with diabetes. For comparison of pre-miR-146a in a podocyte, a suitable control can be pre-miR-146a level in a normal podocyte (e.g., a podocyte from a standard cell line, or from a normal subject, or from the same subject before the subject is diagnosed with diabetes). Alternatively, a suitable control can be a pre-determined value, such as a known value from a database, or from prior investigations or reports, or population average.

In certain embodiments, an increase of pre-miR-146a level in podocytes or glomerular tissue samples of about 25% or more, relative to a suitable control, indicates that said subject is susceptible to developing diabetic nephropathy, or is likely to have a fast progression of diabetic nephropathy. Preferably, the level of pre-miR-146a is increased by about 30% or more, about 40% or more, about 50% or more, about 60% or more, about 70% or more, about 80% or more, about 90% or more, about 100% or more, about 120% or more, about 150% or more, about 200% or more, or about 250% or more, relative to a suitable control.

In certain embodiments, miRNA molecules comprising SEQ ID NO. 2 are used as biomarkers for DN.

Variants of pre-miR-146a, such as pre-microRNA molecules that are at least 85%, at least 90%, or at least 95% identical to SEQ ID NO: 2, can also be used as biomarkers for DN. Fragments of pre-miR-146a can also be used as biomarkers for DN. Preferred fragments or variants comprise the seeding region of miR-146a, or the sequence that binds to the 3′ UTR of Notch-1 or ErbB-4 (as shown in FIG. 2C).

Methods for determining the level of miRNA or pre-miRNA are known in the art. Commonly used methods include, for example, quantitative real time PCR, in situ hybridization, direct sequencing, or mass spectrometry. One exemplary method is using real time PCR with locked nucleic acid (LNA)-based primers (e.g., miRCURY™ LNA microRNA PCR system, Applied Biosystems, Foster City, Calif.; See M. Lunn, et al. Nature Methods, February 2008). In this method, miRNAs are reverse transcribed from total RNA in a sample using miRNA-specific RT primers, and the reverse-transcribed miRNAs are amplified using an LNA-enhanced PCR primer anchored in the miRNA sequence together and a universal PCR primer. Amplified miRNAs are quantitated by a detection agent (such as fluorescence in an SYBR Green assay).

An alternative LNA-based method for quantifying miRNA or pre-miRNA is a direct miRNA assay, as described by L. Neely, et al. Nature Methods, Vol. 3, No. 1, January 2006 (published online Dec. 20, 2005). In this method, two spectrally distinguishable fluorescent LNA-DNA oligonucleotide probes are hybridized to the miRNA of interest, and the tagged molecules are directly counted using single-molecule detection, such as laser-induced fluorescence (LIF) or fluorescence correlation spectroscopy.

Quantifying miRNAs or pre-miRNA using a modification of the Invader assay is described by H. Allawi, et al. (RNA (2004), 10: 1153-1161). In this assay, invasive and probe oligonucleotides are annealed to the miRNA target to form an overlap-flap structure that is a substrate for a structure-specific 5′ nuclease (Cleavase). The non-complementary 5′ flap of the probe is released by cleavage. In a secondary reaction to generate quantifiable signals, a secondary overlap-flap structure is formed by hybridizing both the released 5′ flap and a FRET oligonucleotide to a secondary reaction template. Cleavage between the fluorophore and quencher of the FRET oligonucleotide produces a fluorescent signal which can be quantitated. A 2′-O-methyl arrestor oligonucleotide complementary to the probe sequesters any uncleaved probes so they cannot bind to the secondary reaction template. Because of the small size of miRNAs, the original mRNA assay has been modified to include structures derived from the invasive and probe oligonucleotides in the primary reaction to form a dumbbell-like structure from the 5′ flap is cleaved.

The level of a mature microRNA may be indirectly determined by measuring the level of the immature or unprocessed microRNA. Whether the mature or immature form of a microRNA is measured depends on the detection method, such as which primer or probe is used in the method. Suitable detection methods are known and can be implemented by persons of ordinary skill in the art.

B. Notch-1

Notch-1 a member of the Notch family. Four mammalian Notch homologs have been identified and are designated Notch-1, Notch-2, Notch-3 and Notch-4. Sequence of human Notch-1 (also known as Notch gene homolog 1 and TAN-1) is can be found in public database (accession no. NP_060087 in GenBank). Notch-1 is expressed at very low level in a normal podocyte.

In one aspect, the invention provides a method for assessing a subject's susceptibility to developing diabetic nephropathy, or the progression of diabetic nephropathy in a subject, comprising determining the level of Notch-1 in glomerulus tissue, and/or in one or more podocytes of said subject. An increased level (e.g., expression level, or activity level) of Notch-1, as compared to a suitable control, indicates that the subject is susceptible to developing diabetic nephropathy, or is likely to have a fast progression of renal decline (e.g., fast progression to end-stage renal disease). In certain embodiments, the subject is diagnosed with diabetes.

Suitable controls include level of Notch-1 in a normal podocyte, a glomerular tissue sample from a normal subject, or a predetermined value, as described above.

In certain embodiments, an increase of Notch-1 level in podocytes or glomerular tissue samples of about 1-fold or more, relative to a suitable control, indicates that said subject is susceptible to developing diabetic nephropathy, or is likely to have a fast progression of diabetic nephropathy. Preferably, the level of Notch-1 is increased by about 1.5-fold or more, about 2-fold or more, about 2.5-fold or more, about 3-fold or more, about 4-fold or more, about 5-fold or more, about 6-fold or more, about 7-fold or more, about 8-fold or more, about 9-fold or more, about 10-fold or more, about 12.5-fold or more, about 15-fold or more, or about 20-fold or more, relative to a suitable control.

The level of Notch-1 can be measured by expression level (such as mRNA level, protein level), activity level, or other quantity reflected in or derivable from the gene or protein expression data. For example, the mRNA level of Notch-1 may be measured using quantitative RT-PCR technology that is well known in the art. Typically, RNA (either total RNA or mRNA) is isolated from cells or tissues of interest and is reverse transcribed to yield cDNA. Labeling is usually performed during reverse transcription by incorporating a labeled nucleotide in the reaction mixture. Although various labels can be used, most commonly the nucleotide is conjugated with the fluorescent dyes Cy3 or Cy5. For example, Cy5-dUTP and Cy3-dUTP can be used.

The level of Notch-1 may also be measured by protein level using any art-known method. Traditional methodologies for protein quantification include 2-D gel electrophoresis, mass spectrometry and antibody binding. Typically methods for assaying target protein levels in a biological sample include antibody-based techniques, such as immunoblotting (western blotting), immunohistological assay, enzyme linked immunosorbent assay (ELISA), radioimmunoassay (RIA), or protein chips. Gel electrophoresis, immunoprecipitation and mass spectrometry may be carried out using standard techniques, for example, such as those described in Molecular Cloning A Laboratory Manual, 2nd Ed., ed. by Sambrook, Fritsch and Maniatis (Cold Spring Harbor Laboratory Press: 1989), Harlow and Lane, Antibodies: A Laboratory Manual (1988 Cold Spring Harbor Laboratory), G. Suizdak, Mass Spectrometry for Biotechnology (Academic Press 1996), as well as other references cited herein.

Level of Notch-1 also be measured by the activity level, e.g., by determining activities of Notch-1 signaling pathway.

C ErbB-4

ErbB-4 is a receptor tyrosine kinase, and is a member of the epidermal growth factor receptor subfamily. ERBB4 is a single-pass type I transmembrane protein with multiple furin-like cysteine rich domains, a tyrosine kinase domain, a phosphotidylinositol-3 kinase binding site and a PDZ domain binding motif. The protein binds to and is activated by neuregulins-2 and -3, heparin-binding EGF-like growth factor and betacellulin. Ligand binding induces a variety of cellular responses including mitogenesis and differentiation. Multiple proteolytic events allow for the release of a cytoplasmic fragment and an extracellular fragment. Sequence of human ERBB4 (also known as HER4) is can be found in public database (accession no. NP_001036064 in GenBank). ErbB-4 is expressed at a very low level in a normal podocyte.

In one aspect, the invention provides a method for assessing a subject's susceptibility to developing diabetic nephropathy, or the progression of diabetic nephropathy in a subject, comprising determining the level of ErbB-4 in glomerulus tissue, and/or in one or more podocytes of said subject. An increased level (e.g., expression level, or activity level) of ErbB-4, as compared to a suitable control, indicates that the subject is susceptible to developing diabetic nephropathy, or is likely to have a fast progression of renal decline (e.g., fast progression to end-stage renal disease). In certain embodiments, the subject is diagnosed with diabetes.

Suitable controls include level of ErbB-4 in a normal podocyte, a glomerular tissue sample from a normal subject, or a predetermined value, as described above.

In certain embodiments, an increase of ErbB-4 level in podocytes or glomerular tissue samples of about 1-fold or more, relative to a suitable control, indicates that said subject is susceptible to developing diabetic nephropathy, or is likely to have a fast progression of diabetic nephropathy. Preferably, the level of ErbB-4 is increased by about 1.5-fold or more, about 2-fold or more, about 2.5-fold or more, about 3-fold or more, about 4-fold or more, about 5-fold or more, about 6-fold or more, about 7-fold or more, about 8-fold or more, about 9-fold or more, about 10-fold or more, about 12.5-fold or more, about 15-fold or more, or about 20-fold or more, relative to a suitable control.

The level of ErbB-4 can be measured by expression level (e.g., mRNA level, protein level), activity level, or other quantity reflected in or derivable from the gene or protein expression data, as described in detail above.

D Other Biomarkers.

The three biomarkers disclosed herein can be used singularly, or in any combination, or in combination with other known biomarkers for diagnosis of renal disease, or assessing the risk, progression, or severity of renal disease, or for identifying subjects who would benefit from the treatment described herein.

Exemplary biomarkers that can be used include, for example, Synpo (synaptopodin), WT1 (Wilms tumor 1), podocin, nephrin, etc.

### 4. Methods of Treatment

In another aspect, the invention provides a method of treating diabetic nephropathy, comprising: administering to a subject a therapeutically effective amount of a therapeutic agent selected from the group consisting of: an inhibitor of ErbB4, an inhibitor of Notch-1, miR-146a (or a fragment or variant thereof), a nucleic acid encoding miR-146a (or a fragment or variant thereof), a nucleic acid encoding pre-miR-146a (or a fragment or variant thereof), an miR-146a mimic, a pre-miR-146a mimic, an agent that can increase the level of miR-146a, and a combination thereof; wherein the subject has been diagnosed with diabetes or diabetes susceptibility, and wherein the subject has a low miR-146a level in the glomerular tissue and/or podocytes in comparison to a suitable control.

In another aspect, the invention provides a method for treating diabetic nephropathy, comprising: administering to a subject who has diabetes or diabetes susceptibility, and who has a low miR-146a level in the glomerular tissue and/or podocytes in comparison to a suitable control, a therapeutically effective amount of a therapeutic agent selected from the group consisting of: an inhibitor of ErbB4, an inhibitor of Notch-1, miR-146a (or a fragment or variant thereof), a nucleic acid encoding miR-146a (or a fragment or variant thereof), a nucleic acid encoding pre-miR-146a (or a fragment or variant thereof), an miR-146a mimic, a pre-miR-146a mimic, an agent that can increase the level of miR-146a, and a combination thereof.

In another aspect, the invention provides a method for treating diabetic nephropathy comprising: (i) determining the level of miR-146a in a glomerular tissue or podocyte sample obtained from a subject having diabetes or diabetes susceptibility; and (ii) when the level of miR-146a in the tissue or podocyte sample of said subject is lower than the level in a suitable control, administering to said subject a therapeutically effective amount of a therapeutic agent selected from the group consisting of: an inhibitor of ErbB4, an inhibitor of Notch-1, miR-146a (or a fragment or variant thereof), a nucleic acid encoding miR-146a (or a fragment or variant thereof), a nucleic acid encoding pre-miR-146a (or a fragment or variant thereof), an miR-146a mimic, a pre-miR-146a mimic, an agent that can increase the level of miR-146a, and a combination thereof.

In another aspect, the invention provides a method for identifying a subject for treatment of diabetic nephropathy, comprising: determining the level of miR-146a in a glomerular tissue or podocyte sample obtained from a subject who has diabetes or diabetes susceptibility; wherein, when the level of miR-146a in the tissue or podocyte sample of said subject is lower than the level in the suitable control, the subject is a candidate for treatment of diabetic nephropathy using an agent selected from the group consisting of: an inhibitor of ErbB4, an inhibitor of Notch-1, miR-146a (or a fragment or variant thereof), a nucleic acid encoding miR-146a (or a fragment or variant thereof), a nucleic acid encoding pre-miR-146a (or a fragment or variant thereof), an miR-146a mimic, a pre-miR-146a mimic, an agent that can increase the level of miR-146a, and a combination thereof.

In certain embodiments, the method further comprises: determining the level of ErbB4 and/or Notch-1 in a glomerular tissue or podocyte sample obtained from said subject, wherein said subject is characterized by (i) a high ErbB4 level in the glomerular tissue or podocyte sample, in comparison to a suitable control; and/or (ii) a high Notch-1 level in the glomerular tissue or podocyte sample in comparison to a suitable control.

In certain embodiments, the subject is a mammalian subject, preferable a human subject.

In certain embodiments, the miR-146a comprises SEQ ID NO: 1.

Subject having diabetes susceptibility are subjects who are at high risk of developing diabetes, such as subjects that have a metabolic syndrome, glucose intolerance, increased insulin resistance, obesity, nephropathy, hypothyroidism, hyperthyroidism, a disorder of the pituitary gland, a disorder of the hypothalamus, a disorder of the pancreas, an appetite and eating disorder, etc.

Notch-1 inhibitors are known in the art. Examples of Notch-1 inhibitors include: (i) gamma-secretase inhibitors (e.g., Merck GSI MK-0752; modified di- or tri-peptide with one to two aromatic hydrocarbon rings; nonsteroidal anti-inflammatory drugs (NSAIDs)); (ii) alpha-secretase inhibitors (e.g., inhibitors that target ADAM-10 and -17); (iii) small-molecule blockers (e.g., YK-4-279 reported by Erkizan et al., A small molecule blocking oncogenic protein EWS-FLI1 interaction with RNA helicase A inhibits growth of Ewing's sarcoma, Nat Med. 2009; 15:750-756; MDM2 antagonists reported by Vassilev et al., In vivo activation of the p53 pathway by small-molecule antagonists of MDM2. Science. 2004; 303:844-848; Na+/H+ antiporter Monensin); (iv) endosomal acidification inhibitors; (v) blocking or NRR (negative regulatory region) antibodies (e.g., antibodies that bind to Notch ligand Delta-like-4); (vi) stapled peptide (e.g., hydrocarbon-stapled peptide SAHM1 reported by Moellering et al., Direct inhibition of the NOTCH transcription factor complex. Nature. 2009; 462:182-188); (vii) delivery of notch-inhibiting genes (e.g., a dominant-negative form of Mastermind-like gene, umb/Numb-like gene, FBXW-7 gene); (viii) delivery of siRNAs, shRNAs, or microRNAs (e.g., miR-326). See, Purow. Adv Exp Med Biol. 2012; 727: 305-319; doi: 10.1007/978-1-4614-0899-4_23. Additional exemplary Notch-1 inhibitors can be found, e.g., in US20100093684, US20030225019, and U.S. Pat. No. 7,888,395, incorporated herein by reference.

Preferably, the Notch-1 inhibitor selectively targets Notch-1 without significantly inhibiting the expression or activity of other Notch family members. Alternatively, the inhibitor causes a greater reduction in Notch-1 expression or activity level, as compared to other Notch family members.

ErbB-4 inhibitors are also known in the art, such as therapeutic antibodies that bind to ErbB-4, or small molecule inhibitors. Several small molecule ErbB inhibitors based on a quinazoline structure, and monoclonal antibodies have been developed, such as Erlotinib, Gefitinib, Lapatinib, and SKLB1206. One example of small molecule ErbB inhibitor is JNJ28871063, which is a nonquinazoline pan-ErbB kinase inhibitor having the following structure:

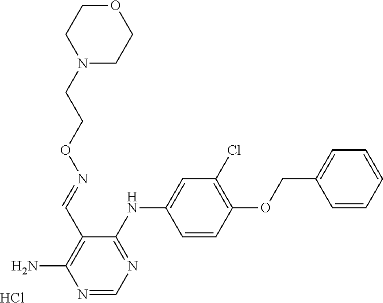 [SMILES: Nc1ncnc(Nc2ccc(OCc3ccccc3)c(Cl)c2)c1/C=N/OCCN1CCOCC1 ; Cl]

Additional exemplary ErbB-4 inhibitors can be found, e.g., in US 20110200618, US 20070092513, and US 20100190964, incorporated herein by reference.

Preferably, the ErbB-4 inhibitor selectively targets ErbB-4 without significantly inhibiting the expression or activity of other ERBB family members. Alternatively, the inhibitor causes a greater reduction in ErbB-4 expression or activity level, as compared to other ERBB family members.

The Notch-1 inhibitors and ErbB-4 inhibitors described herein may be optionally attached to a targeting moiety that direct the delivery of the drug to a specific target. For example, an antibody, or antigen binding fragment thereof that targets podocytes can be attached.

miRNA mimics are typically functionally equivalent to the miRNA duplex that they mimic. It has been found that modification of the sugar backbone can be used to alter stability, hybridization, transport and other properties of the miRNA. For instance, LNA (locked nucleic acid) modifications of the miRNA backbone have been shown to increase the efficiency of silencing of the target mRNA. Similarly, changing the bases sequence by for instance changing an adenosine to an inosine, broadens the target specificity. Of some miRNA duplexes, both strands can be incorporated into RISC, providing two different mature miRNAs. In order to design miRNA mimics with the targeting repertoire of only one of the two alternative mature miRNAs, the sequence of the other strand can be modified such that it no longer acts as the alternative miRNA product.

A miRNA precursor mimic provides a hairpin structure resembling a pre-miRNA hairpin structure as it occurs in nature, so that it serves as a template for the cellular pri-/pre-miRNA processing machinery to allow release of the miRNA duplex in the cell.

miR-146a, pre-miR146a, miR-146a mimics and pre-miR-146a mimics (or a fragment or variant thereof) can be administered directly. Useful fragments and variants include, e.g., a miRNA or miRNA mimic comprising the seeding region of miR-146a, or a miRNA or miRNA mimic comprising the sequence that binds to the 3′ UTR of Notch-1 or ErbB-4 (FIG. 2C).

Alternatively, a nucleic acid encoding miR-146a or pre-miR-146a (or a fragment or variant thereof) can be administered, such as an expression vector that comprises coding sequence for miR-146, pre-miR-146a, or a fragment or variant thereof. The coding sequence may be operably linked to a regulatory element that directs the expression of the coding sequences in a specific type of cell (e.g., podocytes). The choice of vector and/or expression control sequences to which the coding sequence is operably linked to depends on the functional properties desired, e.g., miRNA transcription, and the host cell to be delivered.

Nucleic acid molecules described herein may be attached to a targeting moiety or delivery vehicle that direct the delivery of the drug to a specific target. Because the glomerular capillary wall functions as both a size- and charge-selective barrier, in some cases, it may be desirable to neutralize the negative charge of nucleic acid molecule to facilitate delivery.

miR-146a or pre-miR-146a (or fragments, variants, or mimics thereof) may be delivered to a target cell directly. Alternatively, an expression vector encoding miR-146 or pre-miR-146a (or fragments or variants thereof) may be delivered to a target cell where the miR-146 or pre-miR-146a (or fragments or variants thereof) is expressed. Methods for delivery of oligonucleotides and expression vectors to target cells (e.g., podocytes or glomerulus) are well known in the art and are described briefly below.

Delivery of oligonucleotides and/or expression vectors to a target cell can be achieved in a variety of ways. A transfection agent may be used. A transfection agent, or transfection reagent or delivery vehicle, is a compound or compounds that bind(s) to or complex(es) with oligonucleotides and polynucleotides, and enhances their entry into cells. Examples of transfection reagents include, but are not limited to, cationic liposomes, cationic lipids, polyamines, calcium phosphate precipitates, polycations, histone proteins, polyethylenimine, polylysine, and polyampholyte complexes. Transfection reagents are well known in the art. One exemplary transfection reagent for delivery of miRNA is siPORT™ NeoFX™ transfection agent (Ambion), which can be used to transfect a variety of cell types with miRNA.

Reagents for delivery of miRNAs, pre-miRNAs, miRNA mimics, pre-miRNA mimics, and expression vectors can include, but are not limited to protein and polymer complexes (polyplexes), lipids and liposomes (lipoplexes), combinations of polymers and lipids (lipopolyplexes), and multilayered and recharged particles. Transfection agents may also condense nucleic acids. Transfection agents may also be used to associate functional groups with a polynucleotide. Functional groups can include cell targeting moieties, cell receptor ligands, nuclear localization signals, compounds that enhance release of contents from endosomes or other intracellular vesicles (such as membrane active compounds), and other compounds that alter the behavior or interactions of the compound or complex to which they are attached (interaction modifiers). For delivery in vivo, complexes made with sub-neutralizing amounts of cationic transfection agent may be preferred.

Polycations may be mixed with polynucleotides for delivery to a cell. Polycations are linkers for attaching specific receptors to DNA and as result, DNA/polycation complexes can be targeted to specific cell types.

Polymer reagents for delivery of miRNAs, pre-miRNAs, miRNA mimics, pre-miRNA mimics, and expression vectors may incorporate compounds that increase their utility. These groups can be incorporated into monomers prior to polymer formation or attached to polymers after their formation. A miRNA, pre-miRNA, miRNA mimic, pre-miRNA mimic, or expression vector transfer enhancing moiety is typically a molecule that modifies a nucleic acid complex and can direct it to a cell location (such as tissue cells) or location in a cell (such as the nucleus) either in culture or in a whole organism. By modifying the cellular or tissue location of the complex, the desired localization and activity of the miRNA, pre-miRNA, miRNA mimic, pre-miRNA mimic, or expression vector can be enhanced. The transfer enhancing moiety can be, for example, a protein, peptide, lipid, steroid, sugar, carbohydrate, nucleic acid, cell receptor ligand, or synthetic compound. The transfer enhancing moieties can enhance cellular binding to receptors, cytoplasmic transport to the nucleus and nuclear entry or release from endosomes or other intracellular vesicles.

Nuclear localizing signals can also be used to enhance the targeting of the miRNA, pre-miRNA, miRNA mimic, pre-miRNA mimic, or expression vector into proximity of the nucleus and/or its entry into the nucleus. Such nuclear transport signals can be a protein or a peptide such as the SV40 large Tag NLS or the nucleoplasmin NLS.

Compounds that enhance release from intracellular compartments can cause DNA release from intracellular compartments such as endosomes (early and late), lysosomes, phagosomes, vesicle, endoplasmic reticulum, Golgi apparatus, trans Golgi network (TGN), and sarcoplasmic reticulum and could be used to aid delivery of miRNA-146a, pre-miR-146a, or a fragment, variant, or mimic thereof. Release includes movement out of an intracellular compartment into cytoplasm or into an organelle such as the nucleus. Such compounds include chemicals such as chloroquine, bafilomycin or Brefeldin A1 and the ER-retaining signal (KDEL sequence (SEQ ID NO: 5)), viral components such as influenza virus hemagglutinin subunit HA-2 peptides and other types of amphipathic peptides.

Cellular receptor moieties are any signal that enhances the association of the miRNA, pre-miRNA, miRNA mimic, pre-miRNA mimic, or expression vector with a cell. Enhanced cellular association can be accomplished by either increasing the binding of the polynucleotide or polynucleotide complex to the cell surface and/or its association with an intracellular compartment, for example: ligands that enhance endocytosis by enhancing binding the cell surface.

Dosage can be by a single dose schedule or a multiple dose schedule. In a multiple dose schedule the various doses may be given by the same or different routes. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, about 6 months, about 9 months, about 1 year, about 2 years etc.).

One of skill in the art can determine an effective dose empirically. Methods of determining the most effective means and dosages of administration are well known to those of skill in the art and will vary with the pharmaceutical composition, the target cells, and the subject being treated. Administration can be effected in one dose, continuously or intermittently throughout the course of treatment. Single and multiple administrations can be carried out with the dose level and pattern being selected by the treating physician or researcher.

A “therapeutically effective amount” is an amount that is sufficient to achieve the desired therapeutic or prophylactic effect, such as an amount sufficient to reduce/ameliorate symptoms of a disease that is associated with a disease, prevent or delay the onset or progression of the disease, mitigate the severity of the disease, or protect the cells from further damages.

The dosage administered, as single or multiple doses, to an individual will vary depending upon a variety of factors, including pharmacokinetic properties, the route of administration, patient conditions and characteristics (sex, age, body weight, health, size), extent of symptoms, concurrent treatments, frequency of treatment and the effect desired.

The method of administration include, but are not limited to, oral administration, rectal administration, parenteral administration, subcutaneous administration, intravenous administration, intravitreal administration, intramuscular administration, inhalation, intranasal administration, topical administration, ophthalmic administration, or otic administration.

The ErbB4 inhibitors, Notch-1 inhibitors, miR-146a (or a fragment, variant, or mimic thereof), or a nucleic acid encoding miR-146a or pre-miR-146a (or a fragment or variant thereof) described herein may be administered in any combination, or in combination with other therapeutic agents, such as therapeutic agents for treating diabetes or DN. For example, for treatment of DN, anti-seizure medications, antidepressants, lidocaine patch, or opioids are used for relieving pain. ACE inhibitors, alpha blockers, angiotensin II receptor antagonists, beta blockers, calcium channel blockers and/or diuretics, angiotensinogenases such as renin are used for treating hypertension. For treatment of diabetes, commonly used therapeutics include: metformin (inhibiting hepatic gluconeogenesis), sulfonylureas (increasing insulin secretion), thiazolidinediones (improving adipose lipid metabolism), glucosidase inhibitors (reducing glucose absorption), GLP-1 analogs, amylin analogs, DPP-4 inhibitors (all increase satiety and reduce glucagon), and insulin supplementation. These treatment can be used in any combination with the ErbB4 inhibitors, Notch-1 inhibitors, miR-146a (or a fragment, variant, or mimic thereof), or a nucleic acid encoding miR-146a or pre-miR-146a (or a fragment, variant, or mimic thereof) described herein.

### 5. Pharmaceutical Compositions and Administration

In another aspect, the invention provides a pharmaceutical composition comprising an ErbB4 inhibitor, a Notch-1 inhibitor, miR-146a (or a fragment, variant, or mimic thereof), a nucleic acid encoding miR-146a or pre-miR-146a (or a fragment or variant thereof), or an agent that increases the level of miR-146a.

The pharmaceutical composition may further comprise one or more pharmaceutically acceptable carriers, diluents, or excipients. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Poloxamer (Pluronic F68), any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., N.J. 1991).

The pharmaceutical compositions provided herein may be administered singly or in combination with one or more additional therapeutic agents.

### 6. Kits

In another aspect, the invention provides a kit for assessing a subject's susceptibility to developing diabetic nephropathy, or the progression of diabetic nephropathy in a subject, comprising: (i) a nucleic acid probe that hybridizes to miR-146a or pre-miR-146a; and (ii) a detection agent for determining the level of miR-146a or pre-miR-146a in one or more podocytes, or in a glomerular tissue sample. For example, a fluorescent agent may be used to determine the level of miR-146a or pre-miR-146a, such as the fluorescent agents typically used in a RT-PCR. The nucleic acid probe can comprise nucleotide analogues, such as LNA. Preferably, the nucleic acid probe hybridizes to miR-146a or pre-miR-146a under a stringent condition (e.g., washing in 5×SSC at a temperature of form 50 to 68° C.).

### 7. Screening Methods

In another aspect, the invention provides a method for identifying a candidate agent for treating diabetic nephropathy, comprising: (i) providing a podocyte from a diabetic nephropathy subject, wherein the level of miR-146a in said podocyte is decreased, as compared to the level of miR-146a in a normal podocyte; and (ii) contacting said podocyte with said candidate agent. An increase in the level of miR-146a in the presence of said agent, as compared to the level of miR-146a in the absence of said agent, indicates that said agent is useful for treating diabetic nephropathy.

In another aspect, the invention provides a method for identifying a candidate agent for treating diabetic nephropathy, comprising: (i) providing a normal podocyte (such as podocyte from a standard cell line); (ii) contacting said normal podocyte with (a) a serum sample from a diabetic nephropathy subject; or (b) a high glucose medium; (iii) contacting said podocyte with said candidate agent. An increase in the level of miR-146a in the presence of said agent, as compared to the level of miR-146a in the absence of said agent, indicates that said agent is useful for treating diabetic nephropathy. It was discovered that the serum from a diabetic nephropathy subject cause a decrease in expression of miR-146a in podocytes.

In another aspect, the invention provides a method for identifying a candidate agent for treating diabetic nephropathy, comprising: (i) providing a podocyte from a diabetic nephropathy subject, wherein the level of pre-miR-146a in said podocyte is increased, as compared to the level of pre-miR-146a in a normal podocyte; and (ii) contacting said podocyte with said candidate agent. A decrease in the level of pre-miR-146a in the presence of said agent, as compared to the level of pre-miR-146a in the absence of said agent, indicates that said agent is useful for treating diabetic nephropathy.

In another aspect, the invention provides a method for identifying a candidate agent for treating diabetic nephropathy, comprising: (i) providing a normal podocyte (such as podocyte from a standard cell line); (ii) contacting said normal podocyte with (a) a serum sample from a diabetic nephropathy subject; or (b) high glucose medium; (iii) contacting said podocyte with said candidate agent. A decrease in the level of pre-miR-146a in the presence of said agent, as compared to the level of pre-miR-146a in the absence of said agent, indicates that said agent is useful for treating diabetic nephropathy.

In another aspect, the invention provides a method for identifying a candidate agent for treating diabetic nephropathy, comprising: (i) providing a podocyte from a diabetic nephropathy subject, wherein the expression or activity level of Notch-1 in said podocyte is increased, as compared to the expression or activity level of Notch-1 in a normal podocyte; and (ii) contacting said podocyte with said candidate agent. A decrease in the expression or activity level of Notch-1 in the presence of said agent, as compared to the expression or activity level of Notch-1 in the absence of said agent, indicates that said agent is useful for treating diabetic nephropathy.

In another aspect, the invention provides a method for identifying a candidate agent for treating diabetic nephropathy, comprising: (i) providing a normal podocyte (such as podocyte from a standard cell line); (ii) contacting said normal podocyte with (a) a serum sample from a diabetic nephropathy subject; or (b) high glucose medium; (iii) contacting said podocyte with said candidate agent. A decrease in the expression or activity level of Notch-1 in the presence of said agent, as compared to the expression or activity level of Notch-1 in the absence of said agent, indicates that said agent is useful for treating diabetic nephropathy.

In another aspect, the invention provides a method for identifying a candidate agent for treating diabetic nephropathy, comprising: (i) providing a podocyte from a diabetic nephropathy subject, wherein the expression or activity level of ErbB-4 in said podocyte is increased, as compared to the expression or activity level of ErbB-4 in a normal podocyte; and (ii) contacting said podocyte with said candidate agent. A decrease in the expression or activity level of ErbB-4 in the presence of said agent, as compared to the expression or activity level of ErbB-4 in the absence of said agent, indicates that said agent is useful for treating diabetic nephropathy.

In another aspect, the invention provides a method for identifying a candidate agent for treating diabetic nephropathy, comprising: (i) providing a normal podocyte (such as podocyte from a standard cell line); (ii) contacting said normal podocyte with (a) a serum sample from a diabetic nephropathy subject; or (b) high glucose medium; (iii) contacting said podocyte with said candidate agent. A decrease in the expression or activity level of ErbB-4 in the presence of said agent, as compared to the expression or activity level of ErbB-4 in the absence of said agent, indicates that said agent is useful for treating diabetic nephropathy.

The screening methods can also include the screening of any combination of the biomarkers described herein.

### EXEMPLIFICATION

The invention now being generally described, it will be more readily understood by reference to the following examples, which are included merely for purposes of illustration of certain aspects and embodiments of the present invention, and are not intended to limit the invention.

### Introduction

Diabetic Nephropathy (DN).

DN is a major, progressive complication of diabetes mellitus and the leading cause of end stage renal disease (ESRD) in the US (1). Research over the last several decades has significantly advanced our understanding of the number of molecules and pathways that play critical roles in disease pathogenesis (2-9). Yet, how diabetic mileu causes podocyte injury, foot process effacement and loss is still not completely understood. Additionally, microalbuminuria in diabetic patients is currently the best predictor of progression to ESRD (10, 11). However, not all patients progress to ESRD at the same rate; some progress faster, whereas others are resistant to further decline in renal function and microalbuminuria is not very helpful in stratifying patients in these groups. Thus, biomarkers for earlier, more sensitive diagnosis of patients who are likely to develop nephropathy or progress faster to ESRD are greatly desired, as it will greatly improve the disease management and patient treatment, and improve clinical trial design.

Notch-1.

Notch-1 is a member of a family of four transmembrane proteins that are key developmental proteins (12). Notch pathway is indispensable for renal glomerular and proximal tubule development (13-15). However, the expression of Notch-1 is down-regulated in adult kidneys. Studies show that High Glucose (HG) induces Notch-1 in podocytes. Similarly, kidneys from DN patients, and from experimental models of DN also show high expression of Notch-1 (16, 17). Ligand binding leads to its proteolytic processing, releasing an intracellular domain of Notch1 (ICN) that translocates to the nucleus and mediates expression of a number of target genes. Podocyte specific expression of ICN is sufficient to induce podocyte injury and glomerulosclerosis, suggesting that Notch-1 plays an important role in the pathogenesis of DN. Yet, a detailed molecular mechanism behind how Notch-1 is kept in check in healthy podocytes and how it is upregulated in DN is not clear.

ErbB4.

ErbB4, a receptor tyrosine kinase (RTK), is a member of the epidermal growth factor receptor (EGFR) family of proteins that is expressed in the heart, nervous system, and mammary gland and, like Notch-1, is a key developmental protein (18-21). ErbB4's ligands include neuregulins (NRG) and heparin-binding epidermal growth factor-like growth factor (HB-EGF) (22). ErbB4 is also important for kidney development, where it modulates tubular cell polarity and lumen diameter (21, 22). However, like Notch-1, it shows minimal expression in the adult kidney. A genome-wide association study (GWAS) by the GENIE consortium, with >11,000 type I diabetes (T1D) patients reported their strongest association for DN as a primary phenotype with an intronic SNP in ERBB4 (rs7588550, P=2.1×10⁻⁷), which affects the expression of ErbB4 (23). A link between ErbB4 expression and type 2 diabetes (T2D) has been suggested. This indicates that ErbB4, like Notch-1, may have a significant role in DN. However, to date there are no systematic mechanistic studies confirming whether ErbB4 has a role in DN, and what the related mechanisms are. ErbB4 antagonists are in the clinic to treat a variety of cancers (24).

microRNAs and DN.

MicroRNAs (miRNAs) are a family of non-protein-coding RNAs that are ˜22-nucleotide (22-nt) in length. They sequence-specifically bind to the 3′-UTR of target mRNAs, where they promote mRNA degradation or suppress mRNA translation, thus regulating cellular functions. Because individual miRNAs can target multiple mRNAs in a signaling network, a miRNA can exert control over many cellular pathways. miRNAs are endogenously expressed in the kidney and several have been found to be up- or down-regulated in models of DN (reviewed in (25)) and other renal diseases (26, 27). miR-193a is significantly up-regulated in podocytes in FSGS, where it directly targets WT1 transcripts (27). miRNAs miR-21, -192, -200b, -200c, -216a and -217 are induced in the glomerular mesangial cells in animal models of DN, where a number of them participate in TGFβ-Smad pathway to mediate glomerular damage (25, 28-31). In podocytes and in the glomeruli of animal models of DN, miR-29c is increased under HG conditions (it is also increased in endothelial cells) (32), where it promotes apoptosis by activating Rho kinase via suppression of Spry 1. Unlike other miRNAs, hyperglycemia down-regulates miR-93 in podocytes and in glomeruli of DN animals, thereby increasing VEGF-A expression (33). These studies suggest that diabetic mileu modulates expression of many different miRNAs, and does so differentially in the many cells of the glomerus, where miRNAs play an important role in the pathobiology of DN.

miR-146a.

miR-146a (FIGS. 2A-2B) is a negative regulator of innate immune responses in myeloid cells (34, 35), modulates adaptive immune responses, and has been shown to play central roles in many other cellular functions, including normal hematopoiesis and proliferation of cancer cells (34, 36). miR-146a is also expressed in various endothelial and epithelial cells, although its exact function in these cells is much less clear; oxidative stress/injury of endothelial cells leads to up-regulation of miR-146a, which is shed into exosomes that are then taken up by cardiomyocytes to mediate peripartum cardiomyopathy (37). In another context, miR-146a induction limits pro-inflammatory signaling in endothelial cells (38). In the context of DN, a study found that miR-146a is constitutively expressed in the retinal endothelial cells and is down-regulated by HG (39). It has been reported that miR-146a levels are reduced in the retina, kidney and heart of STZ-induced DN rats. However, it has also been reported that miR-146a levels are increased in the kidney in a model of lupus nephritis (40), thus leaving it unclear as to what its role in the kidney might be.

Amongst its various target mRNAs, miR-146a directly targets Notch-1 (41, 42) and ErbB4 (37, 43) (FIG. 2C). Recent unbiased profiling studies reported that miR-146a is highly expressed in podocytes (44, 45).

### Example 1

### Examine the Mechanistic Basis for how Loss of miR-146a Induces DN In Vivo and if its Effects are Mediated Via Upregulation of Notch-1 and ErbB4

miR-146a Expression is Reduced in the Glomeruli of DN Animals.

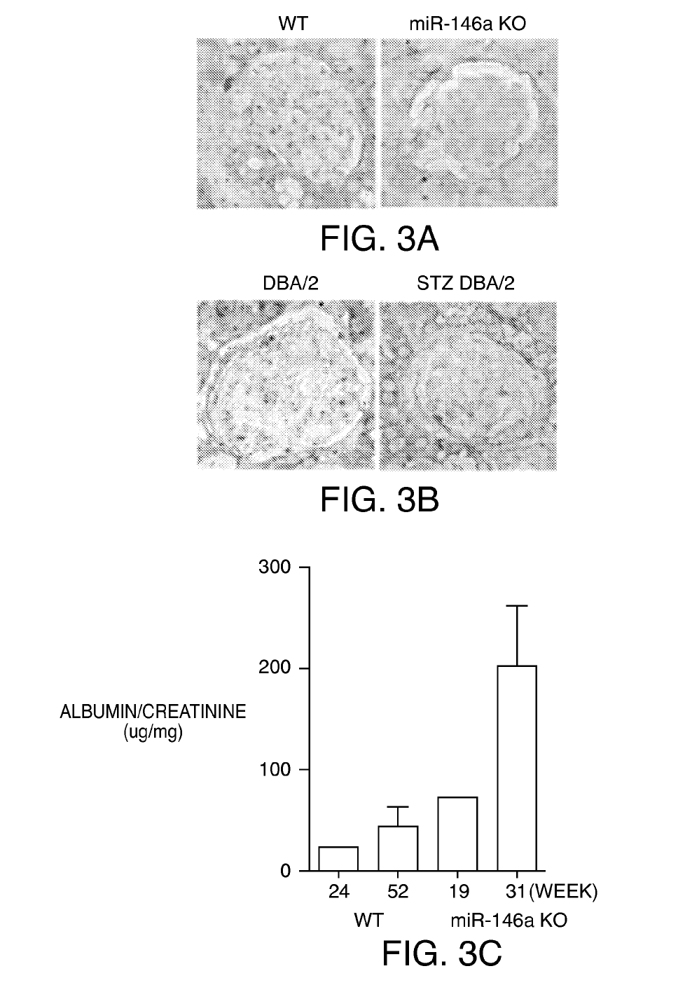

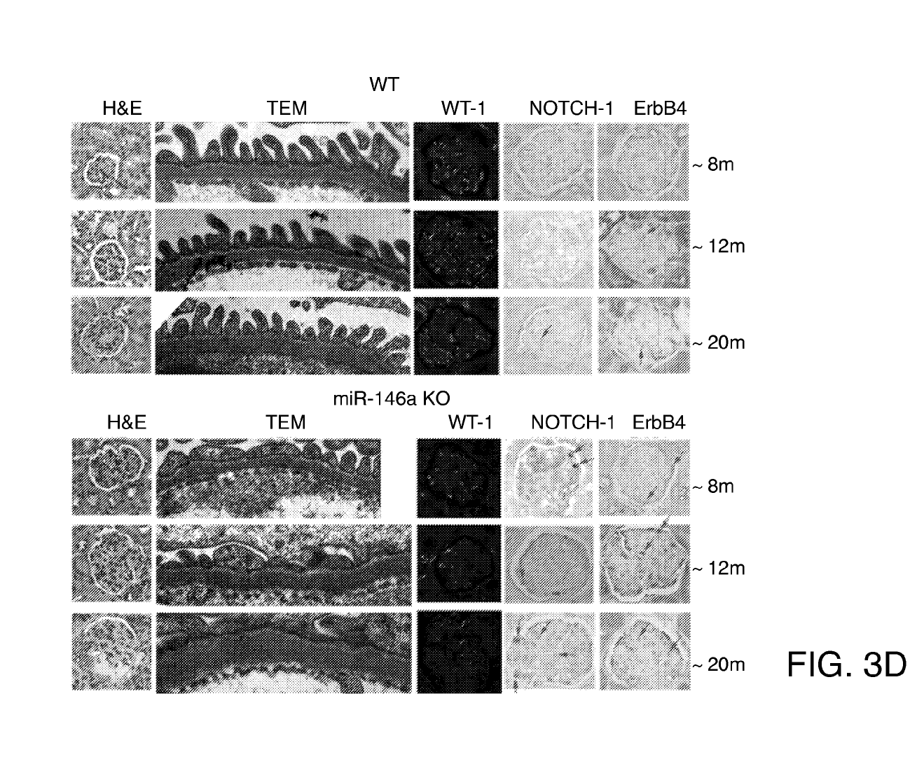

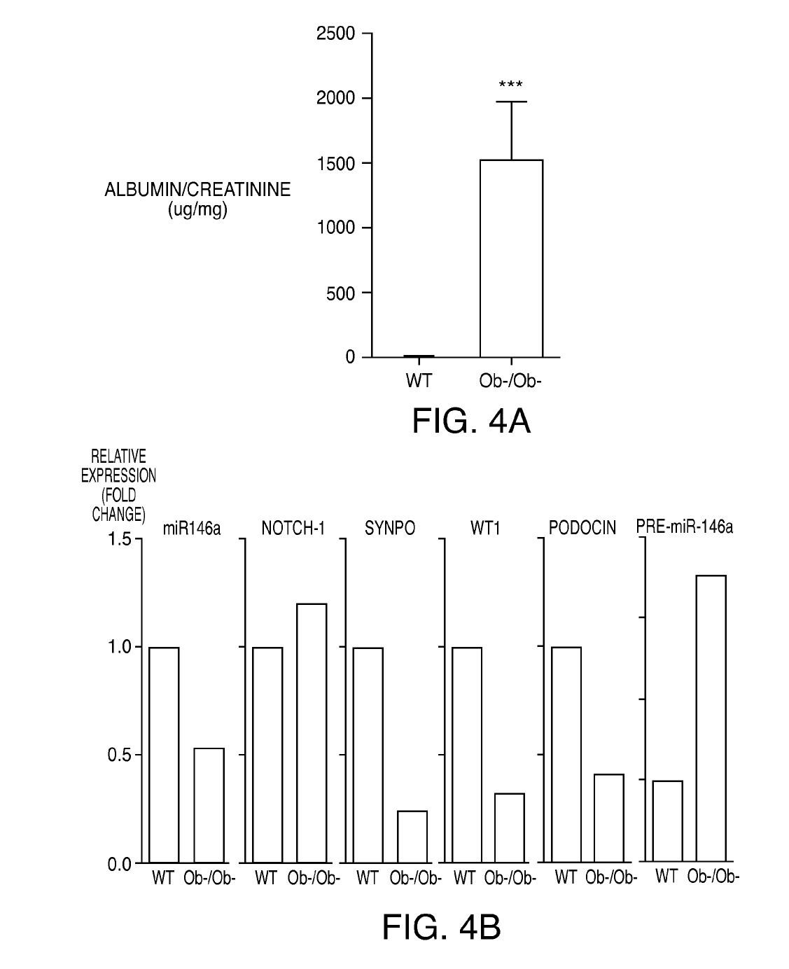

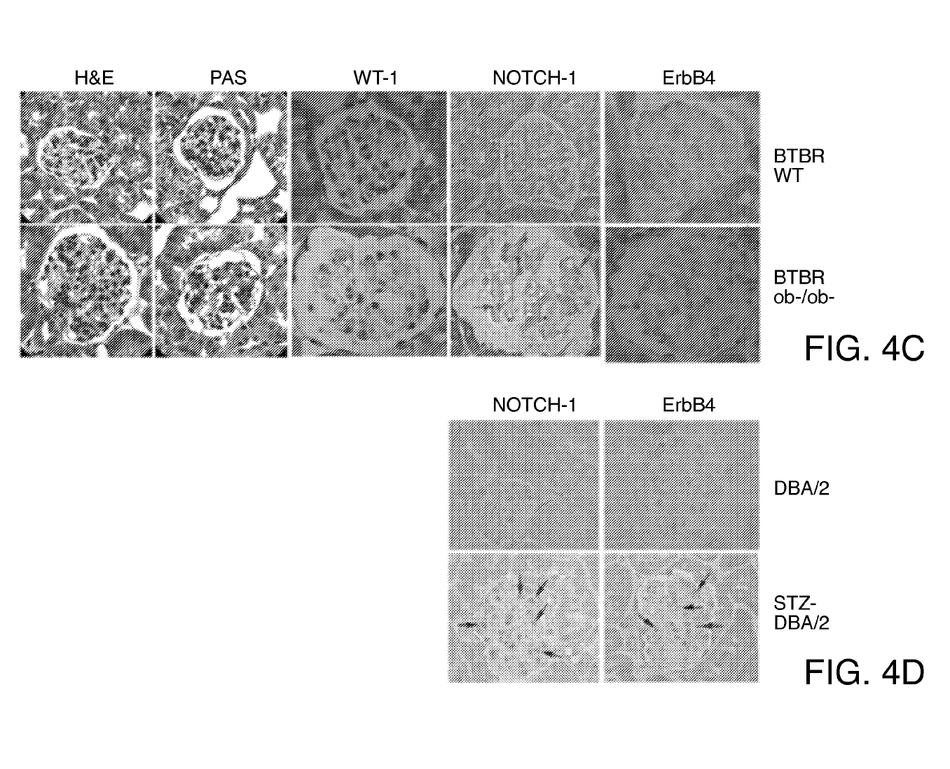

miR-146a is highly expressed in human (44) and mouse podocytes (45). We used in situ hybridization (ISH) to determine miR-146a expression in kidney sections of normal, healthy animals and found that it is expressed in the glomeruli, with highest expression in the podocytes (FIG. 3A). Since microRNAs play a significant role in DN and other glomerular diseases (25). next, we examined miR-146a levels in the glomeruli of DN animals. We utilized two different models; a model of T2DN (the recently described BTBR Ob⁻/Ob⁻ mouse model (46), which demonstrates diabetes induced nephropathy that strongly mimics the pathology of the human disease) and a model of T1DN, the streptozotocin (STZ)-induced DN in DBA/2 mice (47). To our surprise, we found that miR-146a showed reduced expression in the glomeruli of the DN models (by ISH in DBA, FIG. 3B and by qRT-PCR in BTBR Ob−/Ob−, FIG. 4B), as compared to non-DN controls.

miR-146a KO Mice Show Increased DN Pathology with Age.

We investigated levels of albuminuria and the kidney pathology in miR-146a KO animals. miR-146a KO animals showed increasing albuminuria with age (FIG. 3C) suggesting declining renal function. Histological and electron microscopy based analyses of miR-146a KO kidney sections showed significant foot-process effacement, mesangial expansion and loss of podocyte density (visualized using WT-1 immuno-staining), as compared to age-matched B6 WT controls (FIG. 3D), that increased with age. The aged KO animals also showed extensive GBM thickening. It has been reported that miR-146a suppresses expression of Notch-1 (41) and ErbB4 (43). Database searches, using TargetScan (v6.2), picTar and miRBase, also suggest that miR-146a directly targets Notch-1 and ErbB4 (FIG. 2). Therefore, we investigated expression of Notch-1 and ErbB4 in kidney sections, and found that both were highly up-regulated in the podocytes in miR-146a KO kidney sections (FIG. 3D) and in the two DN models (FIGS. 4C-D). Previous studies have shown that upregulation of Notch-1 in podocytes correlates with DN in experimental models and in patients (16, 17), suggesting that miR-146a's function in adult podocytes might be to keep in check such developmental pathways and that, miR-146a down-regulation may lead to transcriptional up-regulation of such proteins, resulting in DN pathology.

Surprisingly, while the levels of mature miR-146a were reduced, we found that the levels of pre-mir-146a increased in DN kidneys (FIG. 4B), suggesting that miR-146a might be regulated in DN at the level of its maturation from pre-miRNA to mature 22-mer, rather than at the transcriptional level.

To investigate if miR-146a plays a role in other glomerular diseases, we also used miR-146a KO animals in models of FSGS and autoimmune nephritis. However, we did not find any increase in LPS-mediated proteinuria in miR-146a KO animals as compared to B6 WT controls (data not shown). Similarly, we did not find any increase in proteinuria in miR-146a KO animals (v/s B6 controls) in a model of anti-GBM nephritis (48) (data not shown), suggesting that glomerular miR-146a may play a role in DN pathology, but not in FSGS or autoimmune GBM nephritis.

Deletion of miR-146a Accelerates STZ Induced DN in Animals.

To further investigate the role of miR-146 in DN, we evaluated miR-146a KO animals in a low-dose STZ-induced DN model (3). We found that, as compared to B6 WT controls, which develop show high albuminuria starting at ˜12-16 weeks of induction (3, 47, 49), the miR-146a KO animals showed increase in proteinuria starting at 6 wks post STZ-treatment (not shown), suggesting that miR-146a deletion greatly accelerates the establishment of DN in animals. The effect of STZ on renal function at later stages in these animals, as well as morphological changes in the glomeruli that are associated with the disease are determined.

### Example 2

### Determine Whether the DN Pathology Accelerates in Animals Lacking miR-146a

A. Examine Whether miR-146a Down-Regulation in Podocytes is a Common Feature of DN and Mediates its Effects Via Notch-1 and ErbB4

To examine whether miR-146a down-regulation in podocytes is a common feature of DNA and mediates its effects via Notch-1 and ErbB4, two types of DN models were selected to validate our findings that suggest that miR-146a deletion leads to accelerated DN.

1. STZ-Induced Models

The experiments utilize the miR-146a KO animals, the miR146a^{+/-} Heterozygotes (Hets) and B6 WT controls. Additionally, podocyte fluorescent reporter mice (podocin-cre driven tomato-GFP reporter mice (45), referred to as Podocin-mtomato here) are crossed with the global miR-146a KO animals (Podocin-mTomato×Mir146^{tm1.1Bal}, referred to as miR146a KO Podocin-mtomato here). The Podocin-mtomato and the miR146a-KO Podocin-mtomato mice are used in these experiments, so that the WT and the miR-146a KO primary podocytes can be easily isolated for molecular and mechanistic analyses. Animals are treated with low dose STZ (3) and the induction of DN is monitored by albuminuria and histolopathological analyses, including TEM based measurements of glomerular ultra-structure (50). miR-146a expression levels in the podocytes are determined using ISH (37, 51) and qRT-PCR after 4, 8, 12 and 16 weeks of STZ to determine when during the disease miR-146a levels decrease, as well as the time-course of the miR-146a expression level changes.

The use of podocin-mtomato miR146a KOs helps to define podocyte-specific changes, given that the miR-146a is globally suppressed in these animals. To confirm that suppression of miR-146a up-regulates Notch-1 and ErbB4 in podocytes in vivo, qRT-PCR and western blotting are used on the isolated glomeruli, as well as histochemical and pathology analyses on the kidney tissues of the animals. The levels of slit-diaphram proteins (such as Nephrin, synaptopodin, podocin), other podocyte proteins (such as WT-1), and proteins in the Notch-1 (12, 14, 15) and ErbB4 pathways (19-22, 52-54) are also quantified. The roles of other members of EGFR family are also investigated by quantifing their levels using qRT-PCR. Expression changes associated with additional genes identified from gene-expression analyses of isolated podocytes from the WT, healthy controls and the DN mice are validated in the tissue.

2. Genetic Models

Data with a genetic model of T2DN (BTBR Ob⁻/Ob⁻) showed that DN in these animals is associated with reduction in miR-146a levels in the glomeruli. Levels of miR-146a are examined in these animals at various stages of the disease (e.g.; 3-4 wks old (pre-nephropathy), 6 wks (early nephropathy) as well as in the 8 wk and 12 wk old homozygous animals as compared to age matched heterozygous mice (BTBR Ob⁺/Ob⁻, Hets) and the BTBR WT, both of which do not develop DN. Similarly, a genetic T1D DN model, the C57BL/6-Ins2^{Akita}/J (Akita) mice (47) (T1D model), can be used.

B. Determine if miR-146a Overexpression Reduces Notch-1 and ErbB4 in Podocytes In Vivo

Studies transiently overexpress miR-146a in non-treated and STZ-treated animals (B6 WT and DBA/2) using in vivo delivery of a podocin-promoter driven miR-146a expression construct are conducted. It has been shown that delivery of such constructs using Mirus TransIT system provides efficient means for expressing various transcripts in podocytes. Similarly, a previous report also utilized this system for podocyte-specific expression of a miR-30a-expressing construct.

Within 1-3 days of the in vivo delivery, the levels of miR-146a in podocytes or in the isolated glomeruli are quantified using ISH and qRT-PCR, as well as the levels of Notch-1 and ErbB4 to determine how well and how quickly miR-146a over-expression suppresses them in vivo. As an alternative, the miR-146a construct is delivered into a Notch-1 dependent nephropathy model, to demonstrate that miR-146a is upstream of Notch-1. Notch signaling pathway is activated in human immunodeficiency virus-associated nephropathy (HIVAN) as well as in a mouse model (Tg26, (59)). where its blockade significantly improves renal function (59, 60). A Tg26 model is utilized to study if the nephropathy in this model is driven in part by reduction in miR-146a expression in podocytes (by qPCR-based measurement of miR-146 and Notch-1 levels in the isolated glomeruli from these animals and with in situ hybridization).

Cell-based studies can also be used, which can confirm the mechanism in podocytes using in vitro systems. Another approach is to generate a mir-146a knock-in mouse.

C. Determine Whether miR-146a Suppression in Podocytes is Sufficient to Induce DN

To establish that miR-146a plays a role in DN pathogenesis, miR-146a is knocked-down in otherwise healthy WT animals. In vivo delivery of anti-sense oligos (ASO) complementary to miR-146a is used to reduce miR-146a levels in podocytes in live B6 WT and db/db animals and the tissue is analyzed using standard biochemical and histological (IHC, immunofluorescence, TEM) analyses. ASOs can be easily designed and be obtained commercially. ASOs against miR-29c over a 12 week period reduced the expression of its target (miR-29c) in the kidney cortex by at least 50%, and similar results were observed with other miR-targeting ASOs. The miR-146a ASO mediated suppression of miR-146a levels is suggested to increase the rate of development of albuminuria and DN pathology in the animals. In situ hybridization confirms the reduction in miR-146a levels.

While the knock-down using this technique is not solely limited to the kidney, it is taken up more by the kidney, and provides additional insights into the role of miR-146a down-modulation in DN.

Transgenic mouse can be generated carrying the miR-146a sponge construct cloned under doxycycline inducible CMV-tet promoter in the ROSA26 locus using recombinase mediated cassette exchange methodology, where doxycycline administration leads to selective over-expression of the miR-146a sponge in podocytes in adult animals. A podocin promoter-driven miR-146a sponge construct is designed (composed of 10 copies of sequence complementary to miR-146a with mismatches at positions 9-12 to enhance stability) to reduce miR-146a levels in podocytes. microRNA sponges are an efficient system to significantly reduce expression of selected miRs in cells.

D. Define the Role of ErbB4 in DN

This example is designed to define the role of ErbB4 in DN, examine whether podocyte specific ErbB4 KO animals are protected from STZ-induced DN, and study if over-expression of ErbB4 or its intracellular domain in podocytes is sufficient to induce DN in animals.

To examine if ErbB4 deficiency in podocytes is protective against DN, Pod-cre×ErbB4(flox/flox) animals, generated by crossing Podocin-cre mice (Pod-Cre) (64) with ErbB4(flox/flox) mice (65) that produce selective deletion of ErbB4 in podocytes (66), are used. It has been reported that ErbB4 knockdown in podocytes did not reduce renal damage in anti-GBM nephritis, indicating this pathway to be specific for DN, as also suggested by preliminary data described herein. The (Pod-cre×ErbB4(flox/flox)) animals are treated with STZ to induce DN and the animals are evaluated as described above.

To determine if ErbB4 gain-of-function in podocytes is sufficient for disease pathogenesis, animals selectively over-expressing ErbB4 in podocytes by crossing Podocin-cre line with available Rosa26 locus-targeted ERBB4 (Rosa26-ErbB4^{flow/flox}) transgenics are developed. Induction of DN in these animals suggests that ErbB4 is sufficient for the pathogenesis of DN, and that podocytic ErbB4 is pathogenic and druggable. Measurement of miR-146a levels in treated animals also defines if there is a feedback mechanism controlling miR-146a maturation. Finally, the mechanism of ErbB4 mediated podocyte damage is addressed in more detail using in vitro assays.

To examine if ligands of ErbB4 (such as Neuregulin-1, heregulin β1, HB-EGF) are up-regulated in DN, and to investigate if ErbB4 signaling is mediated via upregulation of its ligands, expression of ErbB4 ligands is studied in kidney tissue from age-matched, normal controls and in the above described DN animal models. HB-EGF has been shown to be markedly up-regulated in a model of crescentric glomerulopathy, but did not show up-regulation in human DN tissue in that study, suggesting it may not play a role here. Similarly, increased production of glomerular EGF is not very likely to be a contributing factor in DN, as reports found reduced, not increased, expression of EGF in kidney biopsies of DN subjects, including in the tubules (the primary site for their expression). Similarly, while there are limited studies showing the expression of neuregulin-1 in the kidney in DN, neuregulin administration has been suggested to improve other complications of diabetes, including cardiomyopathy and neuropathy, leading to the belief that it may not play a significant role in disease induction in the kidney.

### Example 3

### miR-146a is Highly Expressed in Podocytes and is Down-Regulated by HG and TGFβ. HG and TGFβ Also Up-Regulate miR-146a Target Genes

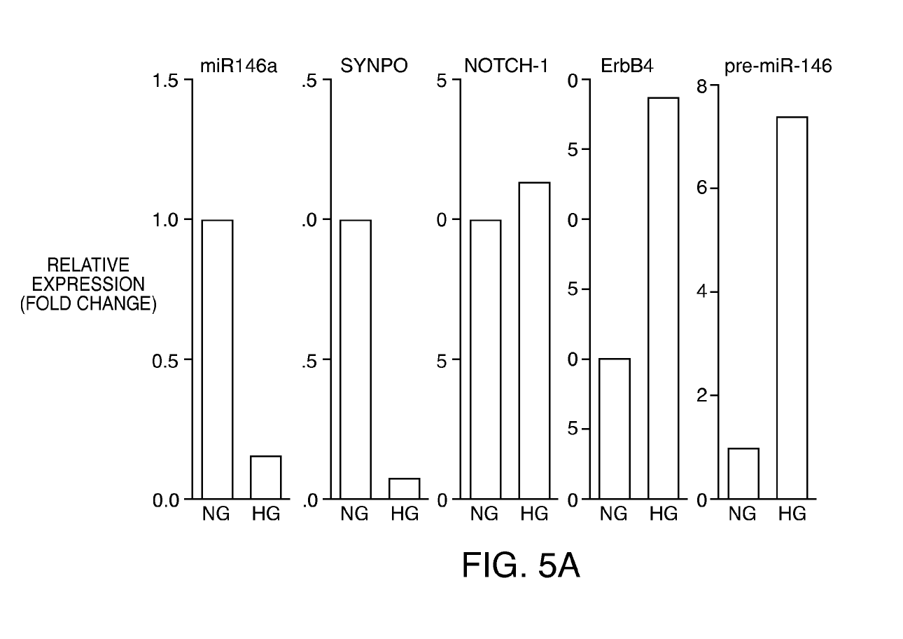

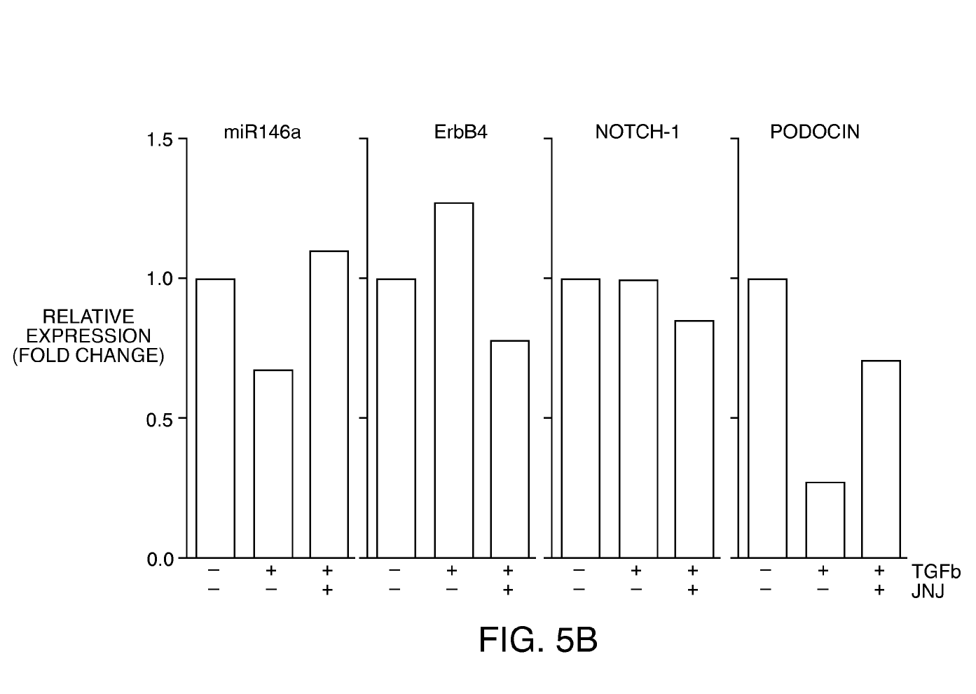

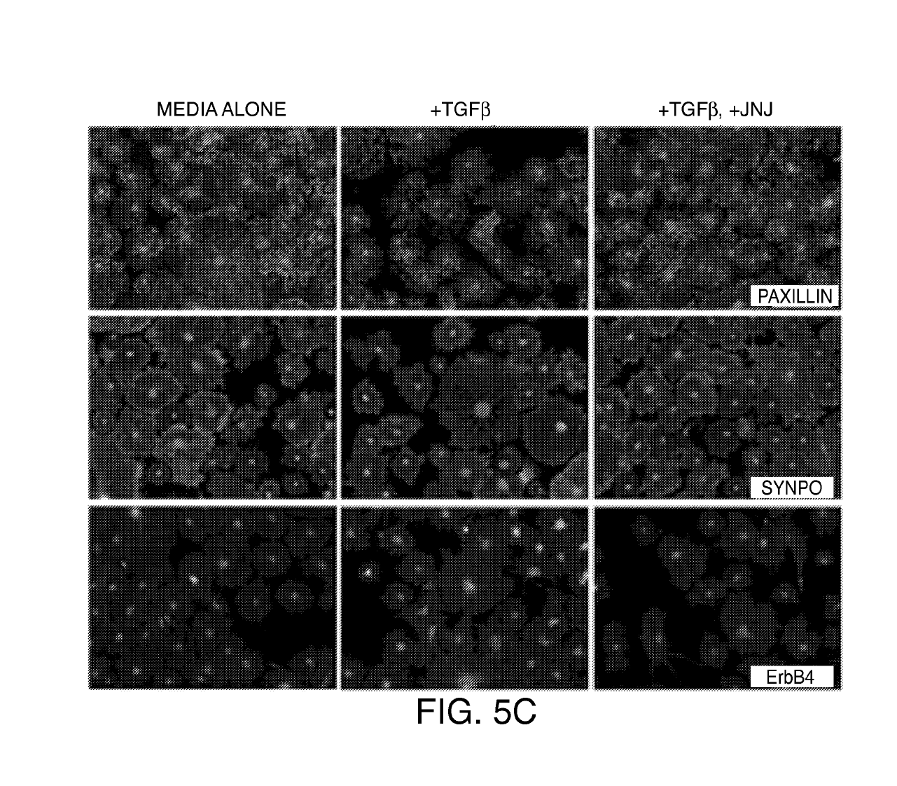

Using quantitative RT-PCR (qPCR), it was found that that miR-146a is constitutively expressed in mouse podocytes (FIG. 5A). Exposure of podocytes to HG or TGFβ (a central player in DN pathogenesis, which has been shown to modulate expression of many other miRs as well as Notch-1) decreased its relative expression levels, whereas iso-osmolar mannitol solution did not. HG and TGFβ also not only showed reduced expression of key podocyte genes, such as Synpo and Podocin, but also increased expression of miR-146a targets Notch-1 and ErbB4, suggesting that diabetic mileu suppresses miR-146a levels, which, in turn, increases the levels of its target transcripts—Notch-1 and ErbB4. Surprisingly, while the levels of mature miR-146a were reduced, it was found that the levels of pre-mir-146a increased, suggesting that miR-146a might be regulated at the level of its maturation from pre-miRNA to mature 22-mer, rather than at the transcriptional level. Using immunofluoresce microscopy it was also found that TGFβ treatment reduced actin fibers, focal adhesions and synpo levels, while increasing the level of ErbB4 in the cells (FIGS. 5A-5C). Finally, given that ErbB4 levels were increased upon HG and TGFb treatment, podocytes were co-treated with ErbB4 inhibitor JNJ28871063 (JNJ), which inhibits ErbB4 with nanomolar affinity (77), revealing that antagonizing ErbB4 reversed the deleterious expression changes induced by TGFβ (FIGS. 5B-5C) at both mRNA and protein level. Lapatinib, inhibitor of EGFR (ErbB1) but only weakly effective on ErbB4, was less effective, suggesting that ErbB4, rather than EGFR, plays a role in podocyte damage in DN.

These studies suggest that the ErbB4 pathway is activated in diabetic podocytes and that clinically available ErbB4 inhibitors may potentially slow down, treat or reverse the DN-induced changes in podocytes.

A. Whether miR-146a Directly Regulate the Expression of Notch-1 and ErbB4 in Podocytes

First, the expression of miR-146a in primary podocytes and its suppression in DN podocytes are validated using isolated primary podocytes from Podocin-mTomato and miR-146a KO Podocin-mTomato mice. Additionally, DN is induced in these animals, using STZ and their podocytes are similarly isolated after 4, 8, 12 and 16 weeks to determine the time-course of the (mature and pre-) miR-146a expression changes with albuminuria (in WT animals) and what the associated global gene expression changes are, including the levels of miR-146a target mRNAs, Notch-1 and ErbB4, as well as additional podocyte proteins (Synpo, Nephrin, WT1, Podocin etc).

To determine if miR-146a is expressed/changed in other glomerular cells, its expression level is measured in both strains of animals and under both conditions (healthy and DN), in non-podocyte cell population isolated during the same podocyte isolation procedure. Confirmation of changes at the protein level is made using western blotting. This analysis also helps define a small subset of the differentially expressed genes that show significant overlap with predicted miR-146a target genes to further define a subset that is regulated by miR-16a in podocytes.

To validate that Notch-1 and ErbB4 are directly targeted by miR-146a in podocytes, as in other cell types, the 3′-UTR luciferase lentiviral constructs are prepared and transfected in podocytes and the luciferase activity is measured in cells cultured in normal media v/s the HG media. Also, the putative miR-146a binding site in the 3′-UTR constructs is mutated to a non-binding sequence to confirm that miR-146a indeed directly targets these UTRs. Additionally, to confirm an interaction between miR-146a and Notch-1 and ErbB4 mRNA, the PAR-CLIP method is used on podocytes from Podocin-mtomato and the Podocin-mtomato-miR146a KO mice.

To further confirm that results are due to miR-146a, cells are co-transfected with miR-146a expression constructs, pre-miR-146a ds RNA or miR-146a mimics (gain of function experiment), or reduce miR-146a levels in cells using antagomirs against miR-146a (silencing experiment) and compare the results with a scrambled antagomir control.

B. Roles of miR-146a, Notch-1, and ErbB4 in Podocyte Injury.

Podocytes cell lines are used stably over-expressing miR-146a (gain-of-function), scrambled control (control), or miR-146a sponge or antagomirs (loss of function). After confirming the mR-146a over-expression or suppression in each of the cell lines, they are treated with HG or TGFβ for various time-points (2 h, 8 h, 24 h, 48 h, 72 h and 1 week). Cellular health (by quantifying F-actin, Synpo, podocin, paxillin, WT-1, Notch-1 and ErbB4 using immunofluorescence, TUNEL staining and mitochondrial integrity) and function (using cell motility, cell adhesion and wound healing assays) are studied. A significant protection of functionally relevant podocyte proteins (actin cytoskeleton, Synpo, Podocin, foot processes (paxillin)), reduction in cellular apoptosis, ROS, mitochondrial damage, caspases and DNA-damage, and maintenance of podocyte functions (integrin-mediated adhesion, reduced motility/wound-healing response) in cells over-expressing miR-146a, but not reduced miR-146a, in response to HG or TGFβ doses, suggests that miR-146a functionally protects healthy podocytes, and that miR-146a suppression sensitizes cells to damage by HG and TGFβ. Furthermore, levels of miR-146a targets, Notch-1 and ErbB4, are analyzed in these cells.

To address the question as to whether it is Notch-1 or ErbB4 that is specifically mediating the effects of miR-146a (or whether both contribute to it), podocytes as well as HEK 293 cells are used. While no ErbB4 specific inhibitors are available, some of the pan-ErbB inhibitors show high affinity and selectivity for ErbB4 (e.g.; JNJ28871063, PD158780, Erlotinib), whereas others, like lapatinib, are much more effective against EGFR v/s ErbB4. These inhibitors are used to block ErbB4. Use of EGFR selective compounds helps determine if some of the functional effects are due to EGFR. The experiments described here help clarify whether miR-146a is directly upstream of Notch-1 and ErbB4 pathways and the role for their cleaved intracellular domains in causing podocyte injury.

While miR-146a modulates ErbB4 at the transcriptional level, ErbB4 has three different modes by which it can signal in a cell. (1) Upon binding its ligand (NRG-1, EGF, HB-EGF), which induces homo- or hetero-dimerization on the cell surface and cross-phosphorylation on tyrosine residues, inducing a signaling cascade involving the ERKMAPK and PI3K/AKT pathways, culminating in gene-expression changes with these indirect signals. (2) GPCR activation (via agonists, such as Angiotensin II) mediated kinase domain activation and subsequent phospho-signaling cascades. GPCRs use intracellular signaling to activate Src kinase, which phosphorylates and activates ErbB4 on tyrosine residues, including at Tyr1188 and Tyr1242 that recruit She 1, whereas ligands activate ErbB4 through extracellular, TACE-mediated mechanism. (3) Via proteolytic processing and release of a cytoplasmic intracellular domain (4ICD), that is mediated by TNFα-converting enzyme (TACE/ADAM 17) and γ-secretase in a fashion similar to the Notch-1 activation pathway, such that 4ICD translocate into the nucleus where it functions as a transcriptional co-activator or co-repressor for a number of transcription factors, such as STAT5. 4ICD can also translocate to mitochondria and promote apoptosis.

C. How does DN Mileu/HG/TGFb Reduce miR-146a Levels in Podocytes?

Unlike myeloid cells, where an inflammatory stimulus increases miR-146a levels via NF-kB activation (34, 35), miR-146a is reduced in DN podocytes. Our data suggest that miR-146a might be regulated at the level of its maturation from pre-miRNA to mature 22-mer, rather than at the transcriptional level.

The process for maturation of microRNAs is incompletely understood. There are two possible mechanisms in the podocytes: a) In addition to Dicer's role in cleaving pre-miRNAs into mature miRs (88), it has been shown that ATM kinase, by enhancing Drosha:pri-mir complex formation, regulates maturation of miRs, including miR-146a, and this has been found to be the case; b) GTPase Git2 (ArfGAP paxillin kinase linker (PKL)/G protein-coupled receptor kinase-interacting protein) regulates miR-146a maturation, by regulating endosome trafficking and fusion and suggests a potential mechanism. Git2 also regulates cell spreading and motility via recruitment of Nck and various other adaptors to focal adhesions. Not wishing to be bound by a particular theory, it is believed that, in response to hyperglycemia, TGFb or diabetic mileu, i) increased ROS will reduce ATM activity, thereby reducing miR-146a maturation and ii) changes in nephrin phosphorylation reduce Nck1/2, which in turn is expected to lead to increased Git2 in the cytosol, where Git2 is known to directly suppress miR-146a maturation. Experiments are carried out to investigate the respective roles for Dicer, Drosha, ATM and Git2 in DN podocytes to establish the mechanism of how miR-146a is reduced in damaged cells.

Given our data, it is validated that HG media and TGFb (in vitro) and diabetic mileu (in vivo) strongly reduce miR-146a expression and induce expression of its target genes Notch-1 and ErbB4 in podocytes. Based on evidence in the literature, miR-146a directly targets Notch-1 and ErbB4 by binding to their 3′-UTR in podocytes and the effects of miR-146a down-regulation in podocytes are mediated by induction of both Notch-1 and ErbB4, that act synergistically. Based on our data, ErbB4 inhibitors have a significant protective effect. Additionally, by defining miR-146a upstream of Notch-1 and ErbB4 in the DN pathway, our data suggest delivery of miR-146a to podocytes as an alternative therapeutic option.

### Example 4

### Determine the Role of Glomerular miR-146a as a Marker to Identify Patients with DN and Rapid Function Decline

miR-146a Expression Levels Inversely Correlate with Glomerular Damage and Proteinuria in DN Patients.

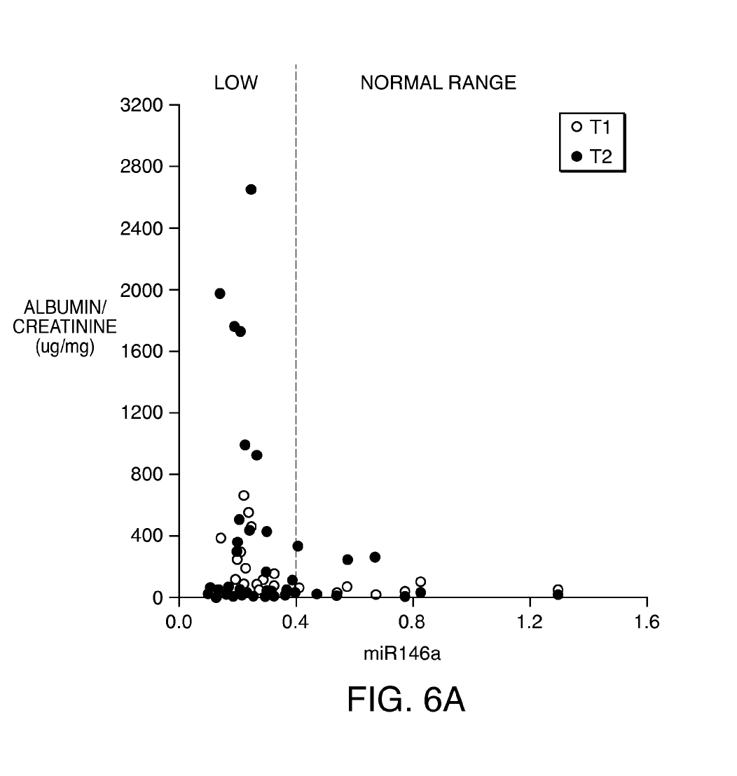

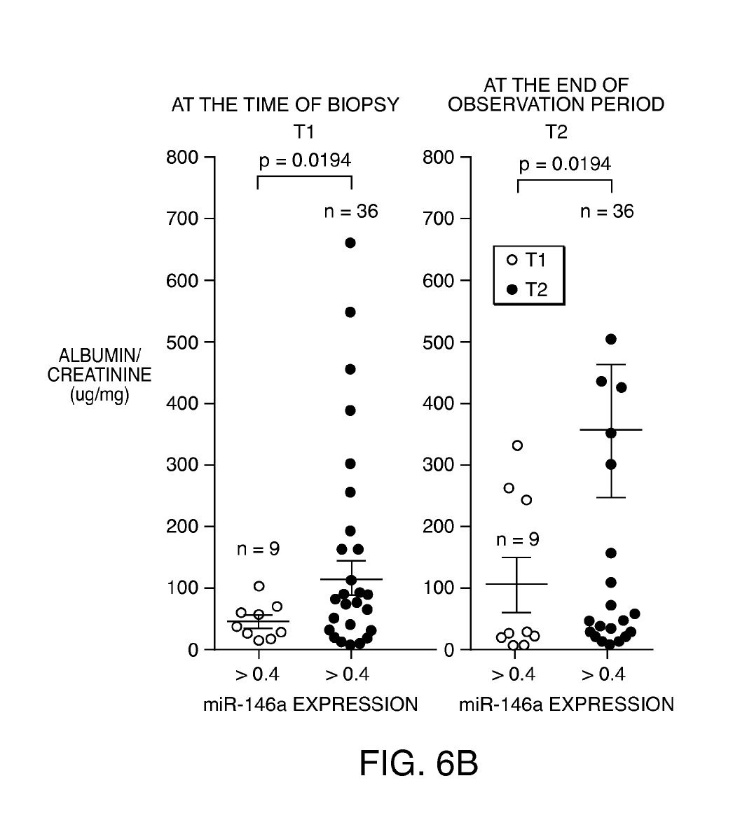

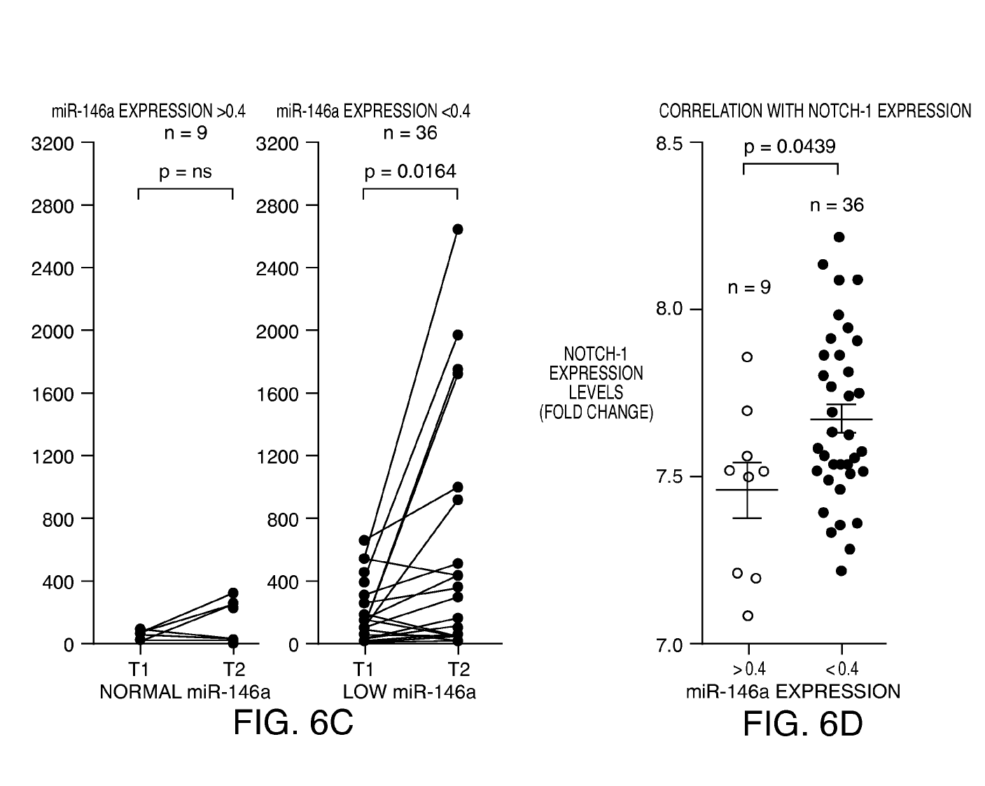

In an experiment, we profiled albumin-to-creatinine ratio (ACR) levels and the levels of miR-146a in isolated glomeruli of kidney biopsies from a cohort of patients with T2DN (the Pima Indian cohort (93, 94)). We found a significant correlation between decreasing relative miR-146a levels and increasing ACR (FIG. 6A). The correlation held when the miR-146a levels from the biopsy were analyzed against ACR measurements from a later, second time-point. The mean±2SD value for miR-146a levels (0.23±0.16=0.4) significantly stratified low miR-146a expressors from relatively “normal” miR-146a levels such that the patients with relative miR-146a expression lower that 0.4 (mean±SEM=113.5±27, red dots), showed significantly higher ACR than patients with miR-146a levels higher than 0.4 (mean±SEM=43.67±9.7, blue dots), with a p-value of 0.0194.

Additionally, this association between low miR-146a and high ACR remained significant with both of the ACR measurements (FIG. 6B). These data suggest that reduction or loss of miR-146a is associated with glomerular damage and proteinuria in DN. Additionally, we performed RNA-seq on micro-dissected glomeruli (glom) and tubulo-interstitium (TI) separately on about 80 kidney biopsy samples from the DN cohort. Preliminary analysis using miRNA clustering method (95) showed that miR-146a cluster was slightly, but significantly, enriched in the glom as compared to the TI tissue (log 2FC (log 2(expression in glom)−log 2(expression in TI)=0.6, p-value: 0.015), suggesting that miR-146a is primarily expressed in the kidney glomeruli in patients and confirmed the findings using the more quantitative qRT-PCR method as described above.

Furthermore, to study if proteinuria and miR-146a expression is associated with glomerular damage, we performed morphometric analyses of the glomeruli from patient biopsies, as described (50). We found a significant correlation between decreased miR-146a levels and increase in the mesangial surface area (p-value: 0.02, correlation: 0.351839). Similarly, we found a significant anti-correlation between miR-146a levels and the filtration surface fraction (p-value: 0.03, correlation: −0.309166). These analyses strongly suggest a correlation between reduced miR-146a expression, glomerular damage and proteinuria in DN patients. Finally, we did not find any down-regulation of miR-146a levels in the glomeruli of FSGS patients (Dontscho Kerjaschki, personal communication), which correlates well with our preliminary experiments with LPS-mediated proteinuria murine model, suggesting that miR-146a may specifically play a role in DN. Further analysis of the ACR data from the time-course measurements showed that there were significant differences in the rate of disease progression between the group of patients with relatively normal levels of miR-146a expression v/s the group with decreased miR-146a levels (FIG. 6C). These data suggest that miR-146a levels may have a predictive value for segregating the fast-progressors from slow-progressors.

Expression of ErbB4 and Notch-1 mRNA, Natural Targets of miR-146a, Increases with Proteinuria in DN Patients and Inversely Correlates with miR-146a Levels.

To further determine if miR-146a levels lead to changes in the down-stream direct targets of this microRNA, we also analyzed the levels of Notch-1 and ErbB4 mRNA in the DN patient biopsies, as described above. The analyses showed that Notch-1 and ErbB4 were both up-regulated in the glomeruli of DN patients and that their expression levels anti-correlated with miR-146a expression (Notch-1, correlation: −0.273; ErbB4, correlation: −0.108). Further analyses showed that decreased miR-146a levels exhibited significant correlation with increased levels of Notch-1 (FIG. 6D) and ErbB4 mRNA (not shown), with relative miR-146a expression <0.4 showing higher expression of these transcripts than samples with miR-146a levels >0.4. These results further suggest that decreased expression of miR-146a results in loss of repression of Notch-1 and ErbB4 and activation of these pathways in glomeruli of patients with DN.

Determine Predictive Potential of Low miR-146a Levels for Identifying Fast-Progressors from Slow-Progressors.

This experiments is used to determine predictive potential of low miR-146a levels for identifying fast-progressors from slow-progressors, by retrospectively associating the baseline miR-146a expression levels (from biopsies) with ACR data across multiple time-points.

As shown in the preliminary data, patients with lower miR146a levels show progression to higher ACR, as compared to patients with levels closer to the normal. This suggests that dynamic profiling of ACR and DN associated changes in DN patients may have a significant correlation with the miR-146a (and Notch-1, ErbB4) expression levels in the kidney biopsies.

To evaluate, patient data from patients who have had multiple ACR measurements and model the correlations between their miR-146a levels and the ACR progression are used. Alternatively, the correlations between patient ErbB4 or Notch-1 levels and the ACR progression are modeled, in case the associations between miR-146a and ACR are found to be not strong enough or to additionally provide support for the miR-146a-ErbB4-Notch-1 signaling in DN.

Collectively, the experiments and data described herein suggest miR-146a (and/or its direct mRNA targets) being used as a diagnostic, early biomarker for DN progression.

### Example 4

### Define the Earliest Time-Point when Glomerular miR-146a Levels can Start Differentiating Between Rapid v/s Slow DN Progressors and Study if Treatment with Clinically Available ErbB4 Inhibitors Reduces the Rate of DN in Experimental Models of DN

A. Experimental Approach

A “mini-trial” in animals is conducted to examine whether low glomerular miR-146a expression can predict faster progression to DN.

To further develop our findings, the following two models are used: 1) a murine model of T1D (D2.B6-Ins2Akita/MatbJ (DBA-Akita) mice (99, 100) and 2) a model for T2D (C57BLKS (BKS)-db/db (db/db) (101)). In both models, while the hyperglycemia leads to renal dysfunction, the rate of renal function decline and the severity of the disease varies between individual animals. Adriamycin (ADR)-induced nephropathy (102), a model of FSGS, is used as a control, where an association between miR-146a decline and rising albuminuria is not expected, based on our preliminary data.

Using these animals (45 per group, a number similar to the number of patients in PD 3.1), serial kidney biopsies (up to four) are performed at various time points, as well as quantitate albuminuria. For heterozygous Akita male mice—the biopsies are at 4, 5, 6 and 7 weeks (these animals show albuminuria at 8-10 wks of age). For db/db animals, the biopsies are at 6, 9, 12 and 15 wks of age (they show glomerular hypertrophy starting at 16 wks of age). The level of miR-146a expression is quantitated using qRT-PCR, as described above and normalize it to housekeeping RNAs (U6). Their glycemia levels, albuminuria, body weight and other characteristics are measured. At the study end point (high albuminuria in each animal), the GFR is measured using FITC-insulin clearance (103), and the kidneys are evaluated by histopathology and TEM to determine glomerular changes, as well as expression levels of miR-146a, Notch-1, ErbB4, Podocin, and WT1 at the study end-point, and determine correlations.

Optionally, correlation analyses between miR-146a levels and glomerular morphology can be performed, to determine if low miR-146a levels better correlate with such changes in the glomeruli, which would still be quite informative and useful. In case that the genetic models turn out to be too homogeneous (too little variability in albuminuria), the STZ-induced DN using WT DBA/2 animals can be used instead.

B. Determine Whether Early Treatment of Animals Showing Relatively Low Glomerular miR-146a Expression with Inhibitors of ErbB4 can Reduce their Progression to DN

This example determines whether treating animals, based on the low miR-146a expression levels in their kidney biopsies early in the disease process, prior to them showing high proteinuria (as described above), would be an effective strategy to reduce their rate of progression to high albuminuria and, thus, treat DN.

Using the same two murine models, as above, a group of animals with biopsy proven low relative miR-146a levels are treated, with daily dose of 10-50 mg/kg ErbB4 inhibitors (PD158780, JNJ28871063 or Erlotinib, LC labs), delivered by oral gavage or i.p. (66), and monitor their albuminuria, glycemia, body weight and overall health over a 4-12 wk period. Their kidneys are examined for features of damage and DN, using standard histochemical techniques. The tissue samples are analyzed for miR-146a, Notch1, ErbB4, Podocin. Synpo, WT1 expression. It is expected that the ErbB4 inhibitors will significantly slow down the rate of albuminuria development in the animals with biopsy-proven low miR-146a levels, and will bring the rate down to at least the rate of progression as shown by relatively normal miR-146a expressors, if not lower.

Published reports also suggest that these inhibitors, which also target EGFR, are efficacious in reducing albuminuria in STZ-induced DN rats (67, 68), anti-GBM nephritis (66) and Ang-II induced albuminuria (69). It is conceivable that our initial doses of the ErbB4 inhibitors may not be optimal, which would necessitate some optimization in a pilot experiment prior to their application in this sub-aim. It is also conceivable that blocking ErbB4, while improving overall proteinuria in the animals, has little effect on the miR-146a levels in the glomeruli. This would validate our model that miR-146a is upstream of ErbB4, yet ErbB4 is the pathogenic protein and, thus, its blockade is beneficial in DN.

### Example 5

### Deletion of miR-146a Accelerates STZ Induced DN in Animals, and is Ameliorated Using ErbB4 Inhibitor

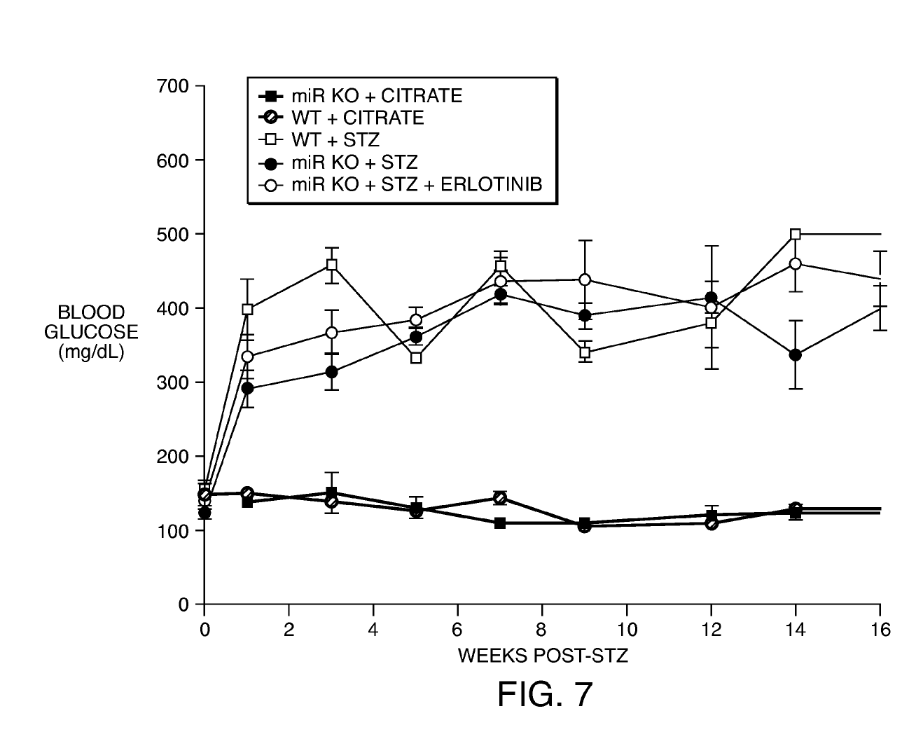

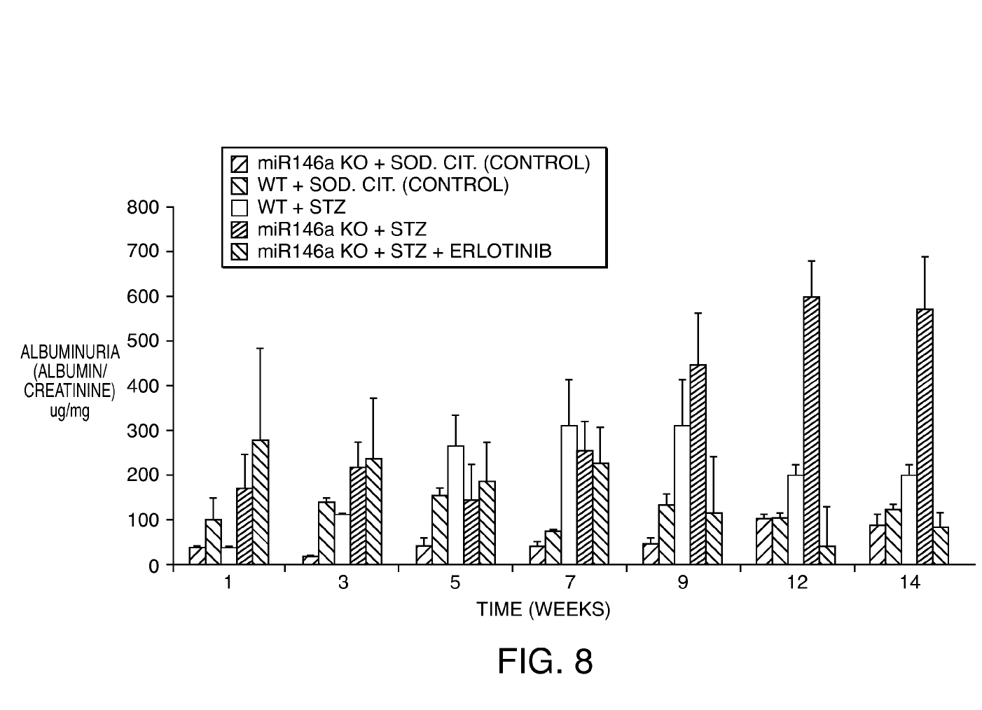

We investigated the role of miR-146 in diabetic nephropathy (DN) using the miR-146a KO animals and a DN mouse model. Streptozotocin (STZ) treatment (125 mg/kg, two doses 3 days apart) induced similar levels of hyperglycemia in both WT and miR146a KO animals (FIG. 7). However, as compared to the WT controls, which did not show high albuminuria until after ˜12-16 wks of induction, the miR-146a KO animals showed increase in proteinuria starting at 6-8 wks post STZ-treatment (FIG. 8), suggesting that miR-146a deletion greatly accelerates the establishment of DN in animals.

To determine if inhibiting ErbB4 using ErbB4 erlotinib would be beneficial and protect animals from DN, we administered Erlotinib (70 mg/kg in saline/Kolliphor, Tween, I.P., daily) in DN prone miR-146a KO animals starting at 4 weeks post-STZ. Albuminuria was assessed and the data showed that erlotinib treatment significantly reduced proteinuria (albuminuria) and protected the animals from the development of DN.

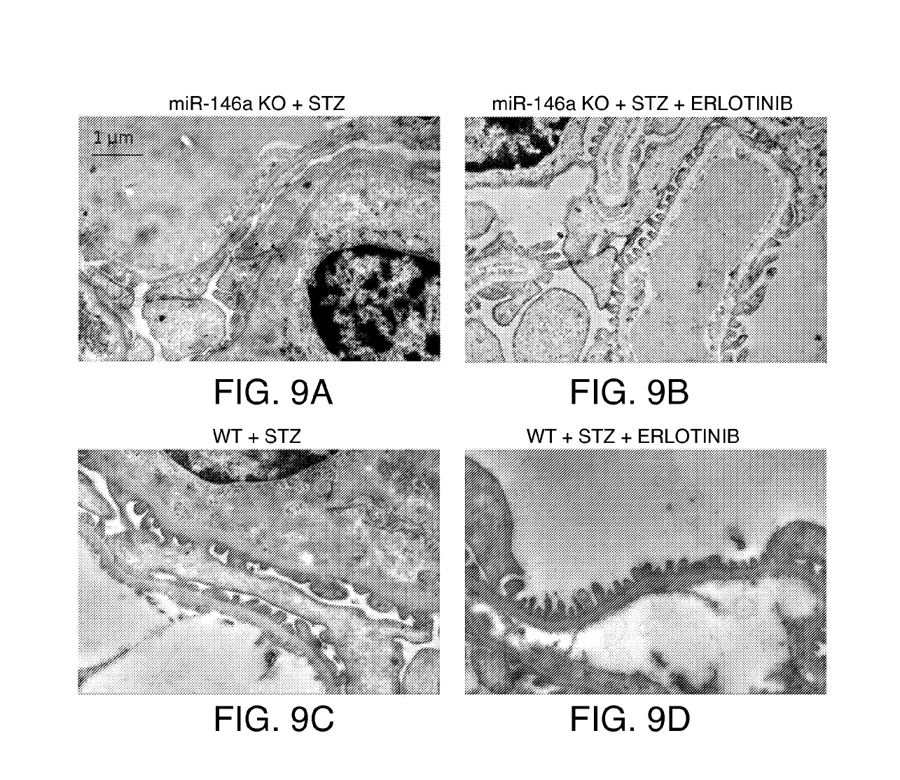

We also analyzed the kidney sections using transmission electron microscopy (TEM) to determine if erlotinib treatment provided protection of podocytes from foot process effacement. TEM was performed according to the standard protocols (Sever, S. et al. J Clin. Invest. 117, 2095-2104 (2007)). TEM analysis showed that erlotinib treatment significantly reduced podocyte foot process effacement in both WT (FIGS. 9C and 9D) and miR-146a KO DN mice (FIGS. 9A and 9B)

These data confirm our findings that ErbB4 pathway is upregulated in the glomeruli in DN and that clinically available ErbB4 inhibitors are potential therapeutics for DN.

### CONCLUSIONS

Based on surprising new findings that microRNA-146a (miR-146a) levels are significantly reduced in the glomeruli of DN patients, and that reduced miR-146a levels correlate with a faster decline in renal function in patients, it is proposed that miR-146a serves as a mechanism and a biomarker to differentiate DN patients into fast and slow progressors, thereby improving clinical management. To-date, miR-146a has primarily been defined by its role in the innate immune system, we now report that it is naturally expressed in podocytes, where its role seems to be to maintain a healthy cell.

Further studies made additional surprising discoveries. The reduced miR-146a expression in isolated glomeruli of DN patients correlated significantly with increased albuminuria and glomerular damage. Genetic miR-146a deficient mice demonstrated increasing albuminuria and DN pathology with age. Additionally, experimental models of DN showed reduced glomerular miR-146a levels with DN pathology. Treatment of podocytes in vitro with high glucose (HG) or TGFb resulted in reduced miR-146a levels. Finally, developmental proteins Notch-1 and ErbB4, which are direct mRNA targets of miR-146a, were unregulated in response to miR-146a down-modulation in each of the previously described findings.

Our findings suggest a novel role for miR-146a in pathogenesis of DN and as a biomarker for disease progression. Given that all three biological targets—miR-146a, Notch-1 and ErbB4—are potentially druggable (using miRNA delivery or inhibitors), this also opens up new possibilities for the treatment of DN patients early in the disease process. Specifically, several ErbB inhibitors, that also target ErbB4, are in the clinic as potent anti-cancer agents. These findings could lead to the application of clinical ErbB4 inhibitors as novel therapeutics for DN. Thus, the studies proposed here have the potential to significantly improve DN patient care and outcomes.

Not wishing to be bound by a particular theory, it is believed that miR-146a is highly expressed in podocytes and that diabetic mileu induces a reduction in miR-146a levels, which results in increased expression of Notch-1 and ErbB4, leading to podocyte damage and diabetic nephropathy, and that glomerular levels of miR-146a can differentiate fast progressors from slow progressors. Described herein are experiments designed to (1) examine the mechanistic basis for how loss of miR-146a induces DN in vivo and if its effects are mediated via upregulation of Notch-1 and ErbB4; (2) study if decrease in miR-146a directly increases levels of its target genes in cultured podocytes and define the mechanism of how ErbB4 causes podocyte damage; (3) determine the role of glomerular miR-146a as a marker to identify patients with DN and rapid function decline; and (4) define the earliest time-point when glomerular miR-146a levels can start differentiating between rapid v/s slow DN progressors and study if treatment with clinically available ErbB4 inhibitors reduces the rate of DN in experimental models of DN.

### REFERENCES

1. USRDS: the United States Renal Data System. American journal of kidney diseases: the official journal of the National Kidney Foundation. 2003; 42(6 Suppl 5): 1-230. PubMed PMID: 14655174.
2. Remuzzi G, Benigni A, Remuzzi A. Mechanisms of progression and regression of renal lesions of chronic nephropathies and diabetes. The Journal of clinical investigation. 2006; 116(2):288-96. doi: 10.1172/JC127699. PubMed PMID: 16453013; PubMed Central PMCID: PMC1359063.
3. Godel M, Hartleben B, Herbach N, Liu S, Zschiedrich S, Lu S, Debreczeni-Mor A, Lindenmeyer M T, Rastaldi M P, Hartleben G, Wiech T, Fornoni A, Nelson R G, Kretzler M, Wanke R, Pavenstadt H, Kerjaschki D, Cohen C D, Hall M N, Ruegg M A, Inoki K, Walz G, Huber T B. Role of mTOR in podocyte function and diabetic nephropathy in humans and mice. The Journal of clinical investigation. 2011; 121(6):2197-209. doi: 10.1172/JCI44774. PubMed PMID: 21606591; PubMed Central PMCID: PMC3104746.
4. Hartleben B, Godel M, Meyer-Schwesinger C, Liu S, Ulrich T, Kobler S, Wiech T. Grahammer F. Arnold S J, Lindenmeyer M T, Cohen C D, Pavenstadt H, Kerjaschki D, Mizushima N, Shaw A S, Walz G, Huber T B. Autophagy influences glomerular disease susceptibility and maintains podocyte homeostasis in aging mice. The Journal of clinical investigation. 2010; 120(4): 1084-96. doi: 10.1172/JCI39492. PubMed PMID: 20200449; PubMed Central PMCID: PMC2846040.
5. Spurney R F, Coffman™. Stressed-out podocytes in diabetes?Journal of the American Society of Nephrology: JASN. 2008; 19(11):2035-7. doi: 10.1681/ASN.2008090955. PubMed PMID: 18842987.
6. Wolf G. Chen S, Ziyadeh F N. From the periphery of the glomerular capillary wall toward the center of disease: podocyte injury comes of age in diabetic nephropathy. Diabetes. 2005; 54(6): 1626-34. PubMed PMID: 15919782.
7. Pagtalunan M E, Miller P L, Jumping-Eagle S, Nelson R G, Myers B D, Rennke H G, Coplon N S, Sun L, Meyer T W. Podocyte loss and progressive glomerular injury in type II diabetes. The Journal of clinical investigation. 1997; 99(2):342-8. doi: 10.1172/JCI119163. PubMed PMID: 9006003; PubMed Central PMCID: PMC507802.
8. Shankland S J. The podocyte's response to injury: role in proteinuria and glomerulosclerosis. Kidney international. 2006; 69(12):2131-47. doi: 10.1038/sj.ki.5000410. PubMed PMID: 16688120.
9. Wiggins R C. The spectrum of podocytopathies: a unifying view of glomerular diseases. Kidney international. 2007; 71(12): 1205-14. doi: 10.1038/sj.ki.5002222. PubMed PMID: 17410103.
10. Mogensen C E, Chachati A, Christensen C K, Close C F, Deckert T, Hommel E, Kastrup J, Lefebvre P, Mathiesen E R, Feldt-Rasmussen B, et al. Microalbuminuria: an early marker of renal involvement in diabetes. Uremia investigation. 1985; 9(2):85-95. PubMed PMID: 3915933.
11. Remuzzi G, Macia M, Ruggenenti P. Prevention and treatment of diabetic renal disease in type 2 diabetes: the BENEDICT study. Journal of the American Society of Nephrology: JASN. 2006; 17(4 Suppl 2):S90-7. doi: 10.1681/ASN.2005121324. PubMed PMID: 16565256.
12. Artavanis-Tsakonas S, Rand M D, Lake R J. Notch signaling: cell fate control and signal integration in development. Science. 1999; 284(5415):770-6. PubMed PMID: 10221902.
13. Cheng H T, Kim M, Valerius M T, Surendran K, Schuster-Gossler K, Gossler A, McMahon A P, Kopan R. Notch2, but not Notch1, is required for proximal fate acquisition in the mammalian nephron. Development. 2007; 134(4):801-11. doi: 10.1242/dev.02773. PubMed PMID: 17229764; PubMed Central PMCID: PMC2613851.
14. Barak H, Surendran K, Boyle S C. The role of Notch signaling in kidney development and disease. Advances in experimental medicine and biology. 2012; 727:99-113. doi: 10.1007/978-1-4614-0899-4_8. PubMed PMID: 22399342.
15. Sirin Y, Susztak K. Notch in the kidney: development and disease. The Journal of pathology. 2012; 226(2):394-403. doi: 10.1002/path.2967. PubMed PMID: 21952830; PubMed Central PMCID: PMC3677191.
16. Niranjan T, Bielesz B, Gruenwald A, Ponda M P, Kopp J B, Thomas D B, Susztak K. The Notch pathway in podocytes plays a role in the development of glomerular disease. Nature medicine. 2008; 14(3):290-8. doi: 10.1038/nm1731. PubMed PMID: 18311147.
17. Waters A M, Wu M Y, Onay T, Scutaru J, Liu J, Lobe C G, Quaggin S E, Piscione T D. Ectopic notch activation in developing podocytes causes glomerulosclerosis. Journal of the American Society of Nephrology: JASN. 2008; 19(6): 1139-57. doi: 10.1681/ASN.2007050596. PubMed PMID: 18337488; PubMed Central PMCID: PMC2396929.
18. Carpenter G. ErbB-4: mechanism of action and biology. Experimental cell research. 2003; 284(1):66-77. PubMed PMID: 12648466.
19. Plowman G D, Culouscou J M, Whitney G S, Green J M, Carlton G W, Foy L. Neubauer M G. Shoyab M. Ligand-specific activation of HER4/p180erbB4, a fourth member of the epidermal growth factor receptor family. Proceedings of the National Academy of Sciences of the United States of America. 1993; 90(5):1746-50. PubMed PMID: 8383326; PubMed Central PMCID: PMC45956.
20. Qiu C, Tarrant M K, Choi S H, Sathyamurthy A, Bose R, Banjade S, Pal A. Bommann W G, Lemmon M A, Cole P A, Leahy D J. Mechanism of activation and inhibition of the HER4/ErbB4 kinase. Structure. 2008; 16(3):460-7. doi: 10.1016/j.str.2007.12.016. PubMed PMID: 18334220; PubMed Central PMCID: PMC2858219.
21. Veikkolainen V, Naillat F, Railo A, Chi L, Manninen A, Hohenstein P, Hastie N, Vainio S, Elenius K. ErbB4 modulates tubular cell polarity and lumen diameter during kidney development. Journal of the American Society of Nephrology: JASN. 2012; 23(1):112-22. doi: 10.1681/ASN.2011020160. PubMed PMID: 22076439; PubMed Central PMCID: PMC3269924.
22. Zeng F, Zhang M Z, Singh A B, Zent R, Harris R C. ErbB4 isoforms selectively regulate growth factor induced Madin-Darby canine kidney cell tubulogenesis. Molecular biology of the cell. 2007:18(11):4446-56. doi: 10.1091/mbc.E07-03-0223. PubMed PMID: 17761534; PubMed Central PMCID: PMC2043549.
23. Sandholm N, Salem R M, McKnight A J, Brennan E P, Forsblom C, Isakova T, McKay G J, Williams W W, Sadlier D M, Makinen V P, Swan E J, Palmer C, Boright A P, Ahlqvist E, Deshmukh H A, Keller B J, Huang H, Ahola A J, Fagerholm E, Gordin D, Harjutsalo V, He B, Heikkila O, Hietala K, Kyto J, Lahermo P, Lehto M, Lithovius R, Osterholm A M, Parkkonen M, Pitkaniemi J, Rosengard-Barlund M, Saraheimo M, Sarti C, Soderlund J, Soro-Paavonen A, Syreeni A, Thorn L M, Tikkanen H. Tolonen N. Tryggvason K, Tuomilehto J, Waden J, Gill G V, Prior S, Guiducci C, Mirel D B, Taylor A, Hosseini S M, Group D E R, Parving H H, Rossing P, Tarnow L, Ladenvall C, Alhenc-Gelas F, Lefebvre P, Rigalleau V, Roussel R, Tregouet D A, Maestroni A. Maestroni S, Falhammar H, Gu T, Mollsten A, Cimponeriu D, Ioana M, Mota M, Mota E, Serafinceanu C, Stavarachi M, Hanson R L, Nelson R G, Kretzler M, Colhoun H M, Panduru N M, Gu H F, Brismar K. Zerbini G, Hadjadj S, Marre M, Groop L, Lajer M, Bull S B, Waggott D, Paterson A D, Savage D A, Bain S C, Martin F, Hirschhorn J N, Godson C, Florez J C. Groop P H, Maxwell A P. New susceptibility loci associated with kidney disease in type 1 diabetes. PLoS genetics. 2012; 8(9):e1002921. doi: 10.1371/journal.pgen.1002921. PubMed PMID: 23028342; PubMed Central PMCID: PMC3447939.
24. Ciardiello F, Tortora G. EGFR antagonists in cancer treatment. The New England journal of medicine. 2008; 358(11):1160-74. doi: 10.1056/NEJMra0707704. PubMed PMID: 18337605.
25. Kato M, Park J T, Natarajan R. MicroRNAs and the glomerulus. Experimental cell research. 2012; 318(9):993-1000. doi: 10.1016/j.yexcr.2012.02.034. PubMed PMID: 22421514; PubMed Central PMCID: PMC3334455.
26. Chau B N, Xin C, Hartner J, Ren S, Castano A P, Linn G, Li J, Tran P T, Kaimal V, Huang X, Chang A N, Li S, Kalra A, Grafals M, Portilla D, MacKenna D A, Orkin S H, Duffield J S. MicroRNA-21 promotes fibrosis of the kidney by silencing metabolic pathways. Science translational medicine. 2012:4(121): 121ra 18. doi: 10.1126/scitranslmed.3003205. PubMed PMID: 22344686; PubMed Central PMCID: PMC3672221.
27. Gebeshuber C A, Kornauth C, Dong L. Sierig R, Seibler J, Reiss M, Tauber S, Bilban M, Wang S, Kain R, Bohmig G A, Moeller M J, Grone H J, Englert C, Martinez J, Kerjaschki D. Focal segmental glomerulosclerosis is induced by microRNA-193a and its downregulation of WT1. Nature medicine. 2013; 19(4):481-7. doi: 10.1038/nm.3142. PubMed PMID: 23502960.
28. Zhang Z, Peng H, Chen J, Chen X, Han F, Xu X, He X, Yan N. MicroRNA-21 protects from mesangial cell proliferation induced by diabetic nephropathy in db/db mice. FEBS letters. 2009; 583(12):2009-14. doi: 10.1016/j.febslet.2009.05.021. PubMed PMID: 19450585.
29. Chandrasekaran K, Karolina D S, Sepramaniam S, Armugam A. Wintour E M, Bertram J F, Jeyaseelan K. Role of microRNAs in kidney homeostasis and disease. Kidney international. 2012; 81(7):617-27. doi: 10.1038/ki.2011.448. PubMed PMID: 22237749.
30. Ho J, Kreidberg J A. The long and short of microRNAs in the kidney. Journal of the American Society of Nephrology: JASN. 2012; 23(3):400-4. doi: 10.1681/ASN.2011080797. PubMed PMID: 22302196; PubMed Central PMCID: PMC3294314.
31. Li J Y, Yong T Y, Michael M Z, Gleadle J M. Review: The role of microRNAs in kidney disease. Nephrology. 2010; 15(6):599-608. doi: 10.1111/j.1440-1797.2010.01363.x. PubMed PMID: 20883280.
32. Long J, Wang Y, Wang W, Chang B H, Danesh F R. MicroRNA-29c is a signature microRNA under high glucose conditions that targets Sprouty homolog 1, and its in vivo knockdown prevents progression of diabetic nephropathy. The Journal of biological chemistry. 2011; 286(13): 1837-48. doi: 10.1074/jbc.M110.194969. PubMed PMID: 21310958; PubMed Central PMCID: PMC3064234.
33. Long J, Wang Y, Wang W, Chang B H, Danesh F R. Identification of microRNA-93 as a novel regulator of vascular endothelial growth factor in hyperglycemic conditions. The Journal of biological chemistry. 2010; 285(30):23457-65. doi: 10.1074/jbc.M110.136168. PubMed PMID: 20501654; PubMed Central PMCID: PMC2906336.
34. Zhao J L, Rao D S, Boldin M P, Taganov K D, O'Connell R M, Baltimore D. NF-kappaB dysregulation in microRNA-146a-deficient mice drives the development of myeloid malignancies. Proceedings of the National Academy of Sciences of the United States of America. 2011; 108(22):9184-9. doi: 10.1073/pnas.105398108. PubMed PMID: 21576471; PubMed Central PMCID: PMC3107319.
35. Taganov K D, Boldin M P, Chang K J, Baltimore D. NF-kappaB-dependent induction of microRNA miR-146, an inhibitor targeted to signaling proteins of innate immune responses. Proceedings of the National Academy of Sciences of the United States of America. 2006; 103(33):12481-6. doi: 10.1073/pnas.0605298103. PubMed PMID: 16885212; PubMed Central PMCID: PMC1567904.
36. Boldin M P, Taganov K D, Rao D S, Yang L, Zhao J L, Kalwani M, Garcia-Flores Y, Luong M, Devrekanli A. Xu J, Sun G, Tay J, Linsley P S, Baltimore D. miR-146a is a significant brake on autoimmunity, myeloproliferation, and cancer in mice. The Journal of experimental medicine. 2011; 208(6):1189-201. doi: 10.1084/jem.20101823. PubMed PMID: 21555486; PubMed Central PMCID: PMC3173243.
37. Halkein J, Tabruyn S P, Ricke-Hoch M, Haghikia A, Nguyen N Q, Scherr M, Castermans K, Malvaux L, Lambert V, Thiry M, Sliwa K, Noel A, Martial J A, Hilfiker-Kleiner D, Struman I. MicroRNA-146a is a therapeutic target and biomarker for peripartum cardiomyopathy. The Journal of clinical investigation. 2013; 123(5):2143-54. doi: 10.1172/JC164365. PubMed PMID: 23619365; PubMed Central PMCID: PMC3638905.
38. Cheng H S, Sivachandran N, Lau A, Boudreau E, Zhao J L, Baltimore D, Delgado-Olguin P, Cybulsky M I, Fish J E. MicroRNA-146 represses endothelial activation by inhibiting pro-inflammatory pathways. EMBO molecular medicine. 2013; 5(7):949-66. doi: 10.1002/emmm.201202318. PubMed PMID: 23733368.
39. Feng B, Chen S, McArthur K, Wu Y. Sen S, Ding Q, Feldman R D, Chakrabarti S. miR-146a-Mediated extracellular matrix protein production in chronic diabetes complications. Diabetes. 2011; 60(11):2975-84. doi: 10.2337/db11-0478. PubMed PMID: 21885871; PubMed Central PMCID: PMC3198068.
40. Ichii O, Otsuka S, Sasaki N. Namiki Y, Hashimoto Y, Kon Y. Altered expression of microRNA miR-146a correlates with the development of chronic renal inflammation. Kidney international. 2012; 81(3):280-92. doi: 10.1038/ki.2011.345. PubMed PMID: 21975861.
41. Mei J, Bachoo R, Zhang C L. MicroRNA-146a inhibits glioma development by targeting Notch1. Molecular and cellular biology. 2011; 31(17):3584-92. doi: 10.1128/MCB.05821-11. PubMed PMID: 21730286; PubMed Central PMCID: PMC3165557.
42. Bai Y, Qian C, Qian L, Ma F, Hou J, Chen Y, Wang Q, Cao X. Integrin CD11b negatively regulates TLR9-triggered dendritic cell cross-priming by upregulating microRNA-146a. Journal of immunology. 2012; 188(11):5293-302. doi: 10.4049/jimmunol.1102371. PubMed PMID: 22551553.
43. Horie T, Ono K, Nishi H, Nagao K, Kinoshita M, Watanabe S. Kuwabara Y, Nakashima Y, Takanabe-Mori R, Nishi E, Hasegawa K, Kita T, Kimura T. Acute doxorubicin cardiotoxicity is associated with miR-146a-induced inhibition of the neuregulin-ErbB pathway. Cardiovascular research. 2010; 87(4):656-64. doi: 10.1093/cvr/cvq48. PubMed PMID: 20495188; PubMed Central PMCID: PMC2920811.
44. Landgraf P, Rusu M, Sheridan R, Sewer A, Iovino N, Aravin A, Pfeffer S, Rice A, Kamphorst A O, Landthaler M, Lin C, Socci N D, Hermida L, Fulci V, Chiaretti S, Foa R, Schliwka J, Fuchs U, Novosel A, Muller R U, Schermer B, Bissels U, Inman J, Phan Q, Chien M, Weir D B, Choksi R, De Vita G, Frezzetti D, Trompeter H I, Hornung V, Teng G, Hartmann G, Palkovits M, Di Lauro R, Wernet P, Macino G. Rogler C E, Nagle J W, Ju J, Papavasiliou F N, Benzing T, Lichter P, Tam W, Brownstein M J, Bosio A. Borkhardt A, Russo J J, Sander C, Zavolan M, Tuschl T. A mammalian microRNA expression atlas based on small RNA library sequencing. Cell. 2007; 129(7): 1401-14. doi: 10.1016/j.cell.2007.04.040. PubMed PMID: 17604727; PubMed Central PMCID: PMC2681231.
45. Boerries M, Grahammer F, Eiselein S, Buck M. Meyer C, Goedel M, Bechtel W, Zschiedrich S, Pfeifer D, Laloe D, Arrondel C, Goncalves S, Kruger M, Harvey S J, Busch H, Dengjel J, Huber T B. Molecular fingerprinting of the podocyte reveals novel gene and protein regulatory networks. Kidney international. 2013; 83(6):1052-64. doi: 10.1038/ki.2012.487. PubMed PMID: 23364521.
46. Hudkins K L, Pichaiwong W, Wietecha T, Kowalewska J, Banas M C, Spencer M W, Muhlfeld A, Koelling M, Pippin J W, Shankland S J, Askari B, Rabaglia M E, Keller M P, Attic A D, Alpers C E. BTBR Ob/Ob mutant mice model progressive diabetic nephropathy. Journal of the American Society of Nephrology: JASN. 2010; 21(9):1533-42. doi: 10.1681/ASN.2009121290. PubMed PMID: 20634301; PubMed Central PMCID: PMC3013527.
47. Brosius F C, 3rd, Alpers C E, Bottinger E P, Breyer M D, Coffman T M, Gurley S B, Harris R C, Kakoki M, Kretzler M, Leiter E H, Levi M, McIndoe R A, Sharma K, Smithies O. Susztak K, Takahashi N, Takahashi T, Animal Models of Diabetic Complications C. Mouse models of diabetic nephropathy. Journal of the American Society of Nephrology: JASN. 2009; 20(12):2503-12. doi: 10.1681/ASN.2009070721. PubMed PMID: 19729434.
48. Ophascharoensuk V, Pippin J W, Gordon K L, Shankland S J, Couser W G, Johnson R J. Role of intrinsic renal cells versus infiltrating cells in glomerular crescent formation. Kidney international. 1998; 54(2):416-25. doi: 10.1046/j.1523-1755.1998.00003.x. PubMed PMID: 9690208.
49. Alpers C E, Hudkins K L. Mouse models of diabetic nephropathy. Current opinion in nephrology and hypertension. 2011; 20(3):278-84. doi: 10.1097/MNH.0b013c3283451901. PubMed PMID: 21422926; PubMed Central PMCID: PMC3658822.
50. Weil E J, Lemley K V, Mason C C, Yee B, Jones L I, Blouch K. Lovato T. Richardson M, Myers B D. Nelson R G. Podocyte detachment and reduced glomerular capillary endothelial fenestration promote kidney disease in type 2 diabetic nephropathy. Kidney international. 2012; 82(9): 1010-7. doi: 10.1038/ki.2012.234. PubMed PMID: 22718189; PubMed Central PMCID: PMC3472108.
51. Renwick N, Cekan P, Masry P A, McGeary S E, Miller J B, Hafner M, Li Z, Mihailovic A, Morozov P, Brown M, Gogakos T, Mobin M B, Snorrason E L, Feilotter H E. Zhang X, Perlis C S, Wu H, Suarez-Farinas M, Feng H, Shuda M, Moore P S, Tron V A, Chang Y, Tuschl T. Multicolor microRNA FISH effectively differentiates tumor types. The Journal of clinical investigation. 2013; 123(6):2694-702. doi: 10.1172/JCI68760. PubMed PMID: 23728175; PubMed Central PMCID: PMC3668843.
52. Zeng F, Xu J, Harris R C. Nedd4 mediates ErbB4 JM-a/CYT-1 ICD ubiquitination and degradation in MDCK II cells. FASEB journal: official publication of the Federation of American Societies for Experimental Biology. 2009; 23(6): 1935-45. doi: 10.1096/fj.08-121947. PubMed PMID: 19193720; PubMed Central PMCID: PMC2698660.
53. Kaushansky A, Gordus A, Budnik B A, Lane W S, Rush J, MacBeath G. System-wide investigation of ErbB4 reveals 19 sites of Tyr phosphorylation that are unusually selective in their recruitment properties. Chemistry & biology. 2008; 15(8):808-17. doi: 10.1016/j.chembiol.2008.07.006. PubMed PMID: 18721752; PubMed Central PMCID: PMC2606095.
54. Schulze W X, Deng L, Mann M. Phosphotyrosine interactome of the ErbB-receptor kinase family. Molecular systems biology. 2005; 1:2005 0008. doi: 10.1038/msb4100012. PubMed PMID: 16729043; PubMed Central PMCID: PMC1681463.
55. Faul C, Donnelly M, Merscher-Gomez S, Chang Y H, Franz S, Delfgaauw J, Chang J M, Choi H Y, Campbell K N. Kim K, Reiser J, Mundel P. The actin cytoskeleton of kidney podocytes is a direct target of the antiproteinuric effect of cyclosporine A. Nature medicine. 2008; 14(9):931-8. doi: 10.1038/nm.1857. PubMed PMID: 18724379.
56. Wei C, El Hindi S, Li J, Fornoni A, Goes N, Sageshima J, Maiguel D, Karumanchi S A, Yap H K, Saleem M. Zhang Q, Nikolic B, Chaudhuri A, Daftarian P, Salido E, Torres A, Salifu M, Sarwal M M, Schaefer F, Morath C. Schwenger V, Zeier M, Gupta V, Roth D, Rastaldi M P, Burke G, Ruiz P, Reiser J. Circulating urokinase receptor as a cause of focal segmental glomerulosclerosis. Nature medicine. 2011; 17(8):952-60. doi: 10.1038/nm.2411. PubMed PMID: 21804539.
57. Wei C, Moller C C, Altintas M M, Li J, Schwarz K, Zacchigna S, Xie L, Henger A, Schmid H, Rastaldi M P, Cowan P, Kretzler M, Parrilla R, Bendayan M, Gupta V, Nikolic B, Kalluri R. Carmeliet P, Mundel P, Reiser J. Modification of kidney barrier function by the urokinase receptor. Nature medicine. 2008; 14(1):55-63. doi: 10.1038/nm11696. PubMed PMID: 18084301.
58. Wu J, Zheng C, Fan Y, Zeng C, Chen Z, Qin W, Zhang C, Zhang W, Wang X, Zhu X, Zhang M, Zen K, Liu Z. Downregulation of MicroRNA-30 Facilitates Podocyte Injury and Is Prevented by Glucocorticoids. Journal of the American Society of Nephrology: JASN. 2013. doi: 10.1681/ASN.2012111101. PubMed PMID: 24029422.
59. Rosenstiel P, Gharavi A. D'Agati V, Klotman P. Transgenic and infectious animal models of HIV-associated nephropathy. Journal of the American Society of Nephrology: JASN. 2009; 20(11):2296-304. doi: 10.1681/ASN.2008121230. PubMed PMID: 19497967.
60. Sharma M, Magenheimer L K, Home T, Tamano K N, Singhal P C, Hyink D P, Klotman P E, Vanden Heuvel G B, Fields T A. Inhibition of Notch pathway attenuates the progression of human immunodeficiency virus-associated nephropathy. American journal of physiology Renal physiology. 2013; 304(8):F1127-36. doi: 10.1152/ajprenal.00475.2012. PubMed PMID: 23389453; PubMed Central PMCID: PMC3625839.
61. Krutzfeldt J, Rajewsky N, Braich R, Rajeev K G, Tuschl T, Manoharan M, Stoffel M. Silencing of microRNAs in vivo with ‘antagomirs’. Nature. 2005; 438(7068):685-9. doi: 10.1038/nature04303. PubMed PMID: 16258535.
62. Ebert M S, Neilson J R, Sharp P A. MicroRNA sponges: competitive inhibitors of small RNAs in mammalian cells. Nature methods. 2007; 4(9):721-6. doi: 10.1038/nmeth1079. PubMed PMID: 17694064.
63. Zhu Q, Sun W, Okano K, Chen Y, Zhang N. Maeda T, Palczewski K. Sponge transgenic mouse model reveals important roles for the microRNA-183 (miR-183)/96/182 cluster in postmitotic photoreceptors of the retina. The Journal of biological chemistry. 2011; 286(36):31749-60. doi: 10.1074/jbc.M111.259028. PubMed PMID: 21768104; PubMed Central PMCID: PMC3173082.
64. Moeller M J, Sanden S K, Soofi A, Wiggins R C, Holzman L B. Podocyte-specific expression of cre recombinase in transgenic mice. Genesis. 2003; 35(1):39-42. doi: 10.1002/gene.10164. PubMed PMID: 12481297.
65. Long W, Wagner K U, Lloyd K C, Binart N, Shillingford J M, Hennighausen L, Jones F E. Impaired differentiation and lactational failure of Erbb4-deficient mammary glands identify ERBB4 as an obligate mediator of STAT5. Development. 2003; 130(21):5257-68. doi: 10.1242/dev.00715. PubMed PMID: 12954715.
66. Bollee G, Flamant M, Schordan S, Fligny C, Rumpel E, Milon M, Schordan E, Sabaa N, Vandermeersch S, Galaup A, Rodenas A, Casal I, Sunnarborg S W, Salant D J, Kopp J B, Threadgill D W, Quaggin S E, Dussaule J C, Germain S, Mesnard L, Endlich K, Boucheix C, Belenfant X, Callard P, Endlich N, Tharaux P L. Epidermal growth factor receptor promotes glomerular injury and renal failure in rapidly progressive crescentic glomerulonephritis. Nature medicine. 2011; 17(10):1242-50. doi: 10.1038/nm.2491. PubMed PMID: 21946538; PubMed Central PMCID: PMC3198052.
67. Advani A, Wiggins K J, Cox A J, Zhang Y, Gilbert R E, Kelly D J. Inhibition of the epidermal growth factor receptor preserves podocytes and attenuates albuminuria in experimental diabetic nephropathy. Nephrology. 2011; 16(6):573-81. doi: 10.1111/j.1440-1797.2011.01451.x. PubMed PMID: 21342330.
68. Wassef L, Kelly D J, Gilbert R E. Epidermal growth factor receptor inhibition attenuates early kidney enlargement in experimental diabetes. Kidney international. 2004; 66(5):1805-14. doi: 10.1111/j.1523-1755.2004.00955.x. PubMed PMID: 15496151.
69. Chen J, Chen J K, Nagai K, Plieth D, Tan M, Lee T C, Threadgill D W, Neilson E G, Harris R C. EGFR signaling promotes TGFbeta-dependent renal fibrosis. Journal of the American Society of Nephrology: JASN. 2012; 23(2):215-24. doi: 10.1681/ASN.2011070645. PubMed PMID: 22095949; PubMed Central PMCID: PMC3269185.
70. Lindenmeyer M T, Kretzler M, Boucherot A, Berra S, Yasuda Y, Henger A. Eichinger F, Gaiser S. Schmid H, Rastaldi M P, Schrier R W, Schlondorff D, Cohen C D. Interstitial vascular rarefaction and reduced VEGF-A expression in human diabetic nephropathy. Journal of the American Society of Nephrology: JASN. 2007; 18(6):1765-76. doi: 10.1681/ASN.2006121304. PubMed PMID: 17475821.
71. Li B, Zheng Z, Wei Y, Wang M, Peng J, Kang T, Huang X, Xiao J, Li Y, Li Z. Therapeutic effects of neuregulin-1 in diabetic cardiomyopathy rats. Cardiovascular diabetology. 2011; 10:69. doi: 10.1186/1475-2840-10-69. PubMed PMID: 21798071; PubMed Central PMCID: PMC3170868.
72. Liu X, Gu X, Li Z, Li X, Li H, Chang J, Chen P, Jin J, Xi B, Chen D, Lai D, Graham R M, Zhou M. Neuregulin-1/erbB-activation improves cardiac function and survival in models of ischemic, dilated, and viral cardiomyopathy. Journal of the American College of Cardiology. 2006; 48(7): 1438-47. doi: 10.1016/j.jacc.2006.05.057. PubMed PMID: 17010808.
73. Odiete O, Hill M F, Sawyer D B. Neuregulin in cardiovascular development and disease. Circulation research. 2012; 111(10):1376-85. doi: 10.1161/CIRCRESAHA.112.267286. PubMed PMID: 23104879; PubMed Central PMCID: PMC3752394.
74. George A J, Hannan R D, Thomas W G. Unravelling the molecular complexity of GPCR-mediated EGFR transactivation using functional genomics approaches. The FEBS journal. 2013. doi: 10.1111/febs.12509. PubMed PMID: 23992425.
75. Bottinger E P, Bitzer M. TGF-beta signaling in renal disease. Journal of the American Society of Nephrology: JASN. 2002; 13(10):2600-10. PubMed PMID: 12239251.
76. Susztak K, Raff A C, Schiffer M, Bottinger E P. Glucose-induced reactive oxygen species cause apoptosis of podocytes and podocyte depletion at the onset of diabetic nephropathy. Diabetes. 2006; 55(1):225-33. PubMed PMID: 16380497.
77. Emanuel S L, Hughes T V, Adams M, Rugg C A, Fuentes-Pesquera A, Connolly P J, Pandey N, Moreno-Mazza S, Butler J, Borowski V, Middleton S A, Gruninger R H, Story J R, Napier C, Hollister B, Greenberger L M. Cellular and in vivo activity of JNJ-28871063, a nonquinazoline pan-ErbB kinase inhibitor that crosses the blood-brain barrier and displays efficacy against intracranial tumors. Molecular pharmacology. 2008; 73(2):338-48. doi: 10.1124/mol.107.041236. PubMed PMID: 17975007.
78. Hafner M, Landthaler M, Burger L, Khorshid M, Hausser J, Berninger P, Rothballer A. Ascano M. Jr., Jungkamp A C, Munschauer M, Ulrich A, Wardle G S, Dewell S, Zavolan M, Tuschl T. Transcriptome-wide identification of RNA-binding protein and microRNA target sites by PAR-CLIP. Cell. 2010; 141(1): 129-41. doi: 10.1016/j.cell.2010.03.009. PubMed PMID: 20371350; PubMed Central PMCID: PMC2861495.
79. Mei L, Xiong W C. Neuregulin I in neural development, synaptic plasticity and schizophrenia. Nature reviews Neuroscience. 2008; 9(6):437-52. doi: 10.1038/nrn2392. PubMed PMID: 18478032; PubMed Central PMCID: PMC2682371.
80. Zheng Y, Zhang C, Croucher D R, Soliman M A, St-Denis N, Pasculescu A, Taylor L, Tate S A, Hardy W R, Colwill K, Dai A Y, Bagshaw R, Dennis J W, Gingras A C, Daly R J, Pawson T. Temporal regulation of EGF signalling networks by the scaffold protein She. Nature. 2013; 499(7457): 166-71. doi: 10.1038/nature12308. PubMed PMID: 23846654.
81. Rio C, Buxbaum J D, Peschon J J, Corfas G. Tumor necrosis factor-alpha-converting enzyme is required for cleavage of erbB4/HER4. The Journal of biological chemistry. 2000; 275(14): 10379-87. PubMed PMID: 10744726.
82. Ni C Y, Murphy M P, Golde T E, Carpenter G. gamma-Secretase cleavage and nuclear localization of ErbB-4 receptor tyrosine kinase. Science. 2001; 294(5549):2179-81. doi: 10.1126/science.1065412. PubMed PMID: 11679632.
83. Williams C C, Allison J G, Vidal G A, Burow M E, Beckman B S, Marrero L, Jones F E. The ERBB4/HER4 receptor tyrosine kinase regulates gene expression by functioning as a STAT5A nuclear chaperone. The Journal of cell biology. 2004; 167(3):469-78. doi: 10.1083/jcb.200403155. PubMed PMID: 15534001; PubMed Central PMCID: PMC2172499.
84. Naresh A, Long W, Vidal G A, Wimley W C, Marrero L, Sartor C I, Tovey S, Cooke T G. Bartlett J M, Jones F E. The ERBB4/HER4 intracellular domain 4ICD is a BH3-only protein promoting apoptosis of breast cancer cells. Cancer research. 2006; 66(12):6412-20. doi: 10.1158/0008-5472.CAN-05-2368. PubMed PMID: 16778220.
85. Yamaoka T, Yan F, Cao H, Hobbs S S, Dise R S, Tong W, Polk D B. Transactivation of EGF receptor and ErbB2 protects intestinal epithelial cells from TNF-induced apoptosis. Proceedings of the National Academy of Sciences of the United States of America. 2008; 105(33): 11772-7. doi: 10.1073/pnas.0801463105. PubMed PMID: 18701712; PubMed Central PMCID: PMC2575333.
86. Mill C P, Gettinger K L, Riese D J, 2nd. Ligand stimulation of ErbB4 and a constitutively-active ErbB4 mutant result in different biological responses in human pancreatic tumor cell lines. Experimental cell research. 2011; 317(4):392-404. doi: 10.1016/j.yexcr.2010.11.007. PubMed PMID: 21110957; PubMed Central PMCID: PMC3022118.
87. Pitfield S E, Bryant I, Penington D J, Park G, Riese D J, 2nd. Phosphorylation of ErbB4 on tyrosine 1056 is critical for ErbB4 coupling to inhibition of colony formation by human mammary cell lines. Oncology research. 2006; 16(4):179-93. PubMed PMID: 17120616; PubMed Central PMCID: PMC2788506.
88. Winter J, Jung S, Keller S, Gregory R I, Diederichs S. Many roads to maturity: microRNA biogenesis pathways and their regulation. Nature cell biology. 2009; 11(3):228-34. doi: 10.1038/ncb0309-228. PubMed PMID: 19255566.
89. Liu Y, Liu Q. ATM signals miRNA biogenesis through KSRP. Molecular cell. 2011; 41(4):367-8. doi: 10.1016/j.molcel.2011.01.027. PubMed PMID: 21329874; PubMed Central PMCID: PMC3075113.
90. Figueredo N, Chora A, Raquel H, Pejanovic N, Pedro P, Hartleben B, Neves-Costa A, Moita C, Pedroso D, Pinto A, Marques S, Faridi H, Costa P, Gozzelino R, Zhao J, Soares M, Gama-Carvalho M, Martinez J, Zhang Q, Döring G, Grompe M, Simas J, Huber T, Baltimore D, Gupta V, Green D, Farreira J, Moita L. Anthracyclins induce autophagy-mediated protection from severe sepsis. Immunity. 2013; Accepted for publication.
91. Zhou W, Thiery J P. Loss of Git2 induces epithelial-mesenchymal transition by miR146a-Cnot6L-controlled expression of Zeb1. Journal of cell science. 2013; 126(Pt 12):2740-6. doi: 10.1242/jcs.126367. PubMed PMID: 23591815.
92. Brown M C, Cary L A, Jamieson J S, Cooper J A, Turner C E. Src and FAK kinases cooperate to phosphorylate paxillin kinase linker, stimulate its focal adhesion localization, and regulate cell spreading and protrusiveness. Molecular biology of the cell. 2005; 16(9):4316-28. doi: 10.1091/mbc.E05-02-0131. PubMed PMID: 16000375; PubMed Central PMCID: PMC1196340.
93. Berthier C C, Zhang H, Schin M, Henger A, Nelson R G, Yee B, Boucherot A, Neusser M A, Cohen C D, Carter-Su C, Argetsinger L S, Rastaldi M P, Brosius F C, Kretzler M. Enhanced expression of Janus kinase-signal transducer and activator of transcription pathway members in human diabetic nephropathy. Diabetes. 2009; 58(2):469-77. doi: 10.2337/db08-1328. PubMed PMID: 19017763; PubMed Central PMCID: PMC2628622.
94. Hodgin J B, Nair V, Zhang H, Randolph A, Harris R C, Nelson R G, Well E J, Cavalcoli J D, Patel J M, Brosius F C, 3rd, Kretzler M. Identification of cross-species shared transcriptional networks of diabetic nephropathy in human and mouse glomeruli. Diabetes. 2013; 62(1):299-308. doi: 10.2337/db11-1667. PubMed PMID: 23139354; PubMed Central PMCID: PMC3526018.
95. Farazi T A, Horlings H M, Ten Hoeve J J, Mihailovic A, Halfwerk H, Morozov P, Brown M, Hafner M, Reyal F, van Kouwenhove M, Kreike B, Sic D, Hovestadt V, Wessels L F, van de Vijver M J, Tuschl T. MicroRNA sequence and expression analysis in breast tumors by deep sequencing. Cancer research. 2011; 71(13):4443-53. doi: 10.1158/0008-5472.CAN-11-0608. PubMed PMID: 21586611; PubMed Central PMCID: PMC3129492.
96. Gadegbeku C A, Gipson D S, Holzman L B, Ojo A O, Song P X, Barisoni L, Sampson M G, Kopp J B, Lemley K V, Nelson P J, Lienczewski C C, Adler S G, Appel G B, Cattran D C, Choi M J, Contreras G, Dell K M, Fervenza F C, Gibson K L, Greenbaum L A, Hernandez J D, Hewitt S M, Hingorani S R, Hladunewich M, Hogan M C, Hogan S L, Kaskel F J, Lieske J C, Meyers K E, Nachman P H, Nast C C, Neu A M, Reich H N, Sedor J R. Sethna C B, Trachtman H, Tuttle K R, Zhdanova O, Zilleruelo G E, Kretzler M. Design of the Nephrotic Syndrome Study Network (NEPTUNE) to evaluate primary glomerular nephropathy by a multidisciplinary approach. Kidney international. 2013; 83(4):749-56. doi: 10.1038/ki.2012.428. PubMed PMID: 23325076; PubMed Central PMCID: PMC3612359.
97. Ju W, Eichinger F, Bitzer M. Oh J, McWeeney S, Berthier C C, Shedden K, Cohen C D, Henger A, Krick S, Kopp J B, Stoeckert C J, Jr., Dikman S, Schroppel B, Thomas D B, Schlondorff D, Kretzler M, Bottinger E P. Renal gene and protein expression signatures for prediction of kidney disease progression. The American journal of pathology. 2009; 174(6):2073-85. doi: 10.2353/ajpath.2009.080888. PubMed PMID: 19465643; PubMed Central PMCID: PMC2684173.
98. Ju W, Greene C S, Eichinger F, Nair V, Hodgin J B, Bitzer M, Lee Y S, Zhu Q. Kehata M. Li M, Jiang S, Rastaldi M P, Cohen C D, Troyanskaya O G, Kretzler M. Defining cell-type specificity at the transcriptional level in human disease. Genome research. 2013. doi: 10.1101/gr.155697.113. PubMed PMID: 23950145.
99. Gurley S B, Mach C L, Stegbauer J, Yang J, Snow K P, Hu A, Meyer T W, Coffman T M. Influence of genetic background on albuminuria and kidney injury in Ins2(+/C96Y) (Akita) mice. American journal of physiology Renal physiology. 2010; 298(3):F788-95. doi: 10.1152/ajprenal.90515.2008. PubMed PMID: 20042456; PubMed Central PMCID: PMC2838602.
100. Qi Z, Fujita H, Jin J, Davis L S, Wang Y, Fogo A B, Breyer M D. Characterization of susceptibility of inbred mouse strains to diabetic nephropathy. Diabetes. 2005; 54(9):2628-37. PubMed PMID: 16123351.
101. Sharma K. McCue P, Dunn S R. Diabetic kidney disease in the db/db mouse. American journal of physiology Renal physiology. 2003; 284(6):F1138-44. doi: 10.1152/ajprenal.00315.2002. PubMed PMID: 12736165.
102. Wang Y, Wang Y P, Tay Y C, Harris D C. Progressive adriamycin nephropathy in mice: sequence of histologic and immunohistochemical events. Kidney international. 2000; 58(4):1797-804. doi: 10.1046/j.1523-1755.2000.00342.x. PubMed PMID: 11012915.
103. Qi Z, Whitt I, Mehta A, Jin J, Zhao M, Harris R C, Fogo A B, Breyer M D. Serial determination of glomerular filtration rate in conscious mice using FITC-inulin clearance. American journal of physiology Renal physiology. 2004; 286(3):F590-6. doi: 10.1152/ajprenal.00324.2003. PubMed PMID: 14600035.
104. Godel, M, Hartleben, B, Herbach, N, Liu, S, Zschiedrich, S. Lu. S, Debreczeni-Mor. A, Lindenmeyer. M T, Rastaldi, M P, Hartleben, G, Wiech, T, Fornoni, A. Nelson, R G, Kretzler, M, Wanke, R. Pavenstadt, H, Kerjaschki, D, Cohen, C D, Hall, M N, Ruegg, M A, Inoki, K, Walz, G, Huber, TB: Role of mTOR in podocyte function and diabetic nephropathy in humans and mice. The Journal of clinical investigation, 121: 2197-2209, 2011. PubMed PMID: 21606591; PubMed Central PMCID: PMC3104746.
105. Alpers, C E, Hudkins, K L: Mouse models of diabetic nephropathy. Current opinion in nephrology and hypertension. 20: 278-284, 2011. doi: 10.1097/MNH.0b013e3283451901. PubMed PMID: 21422926; PubMed Central PMCID: PMC 3658822.
106. Brosius, F C, 3rd, Alpers, C E, Bottinger, E P, Breyer, M D, Coffman, T M, Gurley, S B, Harris, R C, Kakoki, M, Kretzler, M, Leiter, E H, Levi, M, McIndoe, R A, Sharma, K, Smithies, O, Susztak, K. Takahashi, N, Takahashi, T, Animal Models of Diabetic Complications, C: Mouse models of diabetic nephropathy. Journal of the American Society of Nephrology: JASN, 20: 2503-2512, 2009. PubMed PMID: 19729434; PubMed Central PMCID: PMC4075053.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents and NCBI Entrez or gene ID sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

### Sequences

| 1. miR-146a: | |
|---|---|
| (SEQ ID NO: 1) | |
| ugagaacuga auuccauggg uu | |
| 2. pre-miR-146a | |
| (SEQ ID NO: 2) | |
| ccgaugugua uccucagcuu ugagaacuga auuccauggg uugugucagu gucagaccuc | 60 |
| ugaaauucag uucuucagcu gggauaucuc ugucaucgu | 99 |
| 3. miR-146a coding sequence: | |
| (SEQ ID NO: 3) | |
| tgagaactga attccatggg tt | |
| 4. pre-miR-146a coding sequence | |
| (SEQ ID NO: 4) | |
| ccgatgtgta tcctcagctt tgagaactga attccatggg ttgtgtcagt gtcagacctc | 60 |
| tgaaattcag ttcttcagct gggatatctc tgtcatcgt | 99 |

1. A method of treating diabetic nephropathy, comprising:
administering to a subject a therapeutically effective amount of a therapeutic agent selected from the group consisting of: an inhibitor of ErbB4, an inhibitor of Notch-1, miR-146a, a nucleic acid encoding miR-146a, a nucleic acid encoding pre-miR-146a, an miR-146a mimic, a pre-miR-146a mimic, an agent that increases the level of miR-146a, and a combination thereof;
wherein said subject has been diagnosed with diabetes or diabetes susceptibility, and wherein said subject has a low miR-146a level in the glomerular tissue and/or podocytes in comparison to a suitable control.

2. The method of claim 1, wherein said subject is further characterized by (i) a high ErbB4 level in the glomerular tissue and/or podocytes, in comparison to a suitable control; and/or (ii) a high Notch-1 level in the glomerular tissue and/or podocytes in comparison to a suitable control.

3. A method for treating diabetic nephropathy, comprising:
administering to a subject who has diabetes or diabetes susceptibility, and who has a low miR-146a level in the glomerular tissue and/or podocytes in comparison to a suitable control, a therapeutically effective amount of a therapeutic agent selected from the group consisting of: an inhibitor of ErbB4, an inhibitor of Notch-1, miR-146a, a nucleic acid encoding miR-146a, a nucleic acid encoding pre-miR-146a, an miR-146a mimic, a pre-miR-146a mimic, an agent that increases the level of miR-146a, and a combination thereof.

4. The method of claim 3, wherein said subject is further characterized by (i) a high ErbB4 level in the glomerular tissue and/or podocytes, in comparison to a suitable control; and/or (ii) a high Notch-1 level in the glomerular tissue and/or podocytes in comparison to a suitable control.

5. The method of claim 1, comprising administering to said subject a therapeutically effective amount of an inhibitor of ErbB4.

6. The method of claim 1, comprising administering to said subject a therapeutically effective amount of an inhibitor of Notch-1.

7. The method of claim 1, comprising administering to said subject a therapeutically effective amount of miR-146a, a nucleic acid encoding miR-146a, a nucleic acid encoding pre-miR-146a, a miR-146a mimic, or a pre-miR146a mimic.

8. The method claim 1, wherein the miR-146a level in the glomerular tissue of said subject is decreased about 50% or more, in comparison to a suitable control.

9. The method of claim 1, wherein the miR-146a level in podocytes of said subject is decreased about 50% or more, in comparison to a suitable control.

10. The method of claim 1, wherein said subject is a human subject.

11. The method of claim 1, wherein said miR-146a comprises SEQ ID NO: 1.

12. A method for treating diabetic nephropathy comprising:
determining the level of miR-146a in a glomerular tissue or podocyte sample obtained from a subject having diabetes or diabetes susceptibility; and
when the level of miR-146a in the tissue or podocyte sample of said subject is lower than the level in a suitable control, administering to said subject a therapeutically effective amount of a therapeutic agent selected from the group consisting of: an inhibitor of ErbB4, an inhibitor of Notch-1, miR-146a, a nucleic acid encoding miR-146a, a nucleic acid encoding pre-miR-146a, an miR-146a mimic, a pre-miR-146a mimic, an agent that increases the level of miR-146a, and a combination thereof.

13. The method of claim 12, further comprising: determining the level of ErbB4 and/or Notch-1 in a glomerular tissue or podocyte sample obtained from said subject, wherein said subject is characterized by (i) a high ErbB4 level in the glomerular tissue or podocyte sample, in comparison to a suitable control; and/or (ii) a high Notch-1 level in the glomerular tissue or podocyte sample in comparison to a suitable control.

14. A method for identifying a subject for treatment of diabetic nephropathy, comprising:
determining the level of miR-146a in a glomerular tissue or podocyte sample obtained from a subject who has diabetes or diabetes susceptibility;
wherein, when the level of miR-146a in the tissue or podocyte sample of said subject is lower than the level in the suitable control, the subject is a candidate for treatment of diabetic nephropathy using an agent selected from the group consisting of: an inhibitor of ErbB4, an inhibitor of Notch-1, miR-146a, a nucleic acid encoding miR-146a, a nucleic acid encoding pre-miR-146a, an miR-146a mimic, a pre-miR-146a mimic, an agent that increases the level of miR-146a, and a combination thereof.

15. The method of claim 14, further comprising: determining the level of ErbB4 and/or Notch-1 in a glomerular tissue or podocyte sample obtained from said subject, wherein said subject is characterized by (i) a high ErbB4 level in the glomerular tissue or podocyte sample, in comparison to a suitable control; and/or (ii) a high Notch-1 level in the glomerular tissue or podocyte sample in comparison to a suitable control.

16. The method of claim 12, comprising administering to said subject a therapeutically effective amount of an inhibitor of ErbB4.

17. The method of claim 12, comprising administering to said subject a therapeutically effective amount of an inhibitor of Notch-1.

18. The method of claim 12, comprising administering to said subject a therapeutically effective amount of miR-146a, a nucleic acid encoding miR-146a, a nucleic acid encoding pre-miR-146a, an miR-146a mimic, or a pre-miR-146a mimic.

19. The method of claim 12, wherein the miR-146a level in a glomerular tissue sample from said subject is decreased about 50% or more, in comparison to a suitable control.

20. The method of claim 12, wherein the miR-146a level in a podocyte sample of said subject is decreased about 50% or more, in comparison to a suitable control.

21. The method of claim 12, wherein said subject is a human subject.

22. The method of claim 12, wherein said miR-146a comprises SEQ ID NO: 1.

23. A method for identifying a candidate agent for treating diabetic nephropathy, comprising:
(a) providing a podocyte from a diabetic nephropathy subject, wherein the level of miR-146a in said podocyte is decreased, as compared to a suitable control; and
(b) contacting said podocyte with said candidate agent;
wherein an increase in the level of miR-146a in the presence of said agent, as compared to the level of miR-146a in the absence of said agent, is indicative that said agent is useful for treating diabetic nephropathy.

24. A method for identifying a candidate agent for treating diabetic nephropathy, comprising:
(a) providing a normal podocyte;
(b) contact said normal podocyte with a serum sample from a diabetic nephropathy subject;
(c) contacting said podocyte with said candidate agent;
wherein an increase in the level of miR-146a in the presence of said agent, as compared to the level of miR-146a in the absence of said agent, is indicative that said agent is useful for treating diabetic nephropathy.

25. The method of claim 23, wherein said miR-146a comprises SEQ ID NO: 1.

26. A kit for assessing a subject's susceptibility to developing diabetic nephropathy, or the progression of diabetic nephropathy in a subject, comprising:
(a) a nucleic acid probe that hybridizes to miR-146a; and
(b) a detection agent for determining the level of miR-146a in one or more podocytes, or in a glomerular tissue sample.

27. The kit of claim 26, wherein said miR-146a comprises SEQ ID NO: 1.

28. The kit of claim 26, wherein said detection agent comprises a fluorescent agent.
